(19) United States
(12) Patent Application Publication
(10) Pub. No.: US 2023/0010850 A1
(43) Pub. Date: Jan. 12, 2023

(54) ORGANIC SOLVENT PRODUCTION VIA DISTILLATION AND DEHYDRATION

(71) Applicant: WHITEFOX TECHNOLOGIES LIMITED, London (GB)

(72) Inventors: Thiago Righi, Calgary (CA); Chenxu Shi, Calgary (CA); Virginia Andrade, Calgary (CA); Stephan BLUM, Calgary (CA)

(73) Assignee: WHITEFOX TECHNOLOGIES LIMITED, London (GB)

(21) Appl. No.: 17/862,780

(22) Filed: Jul. 12, 2022

Related U.S. Application Data
(60) Provisional application No. 63/220,837, filed on Jul. 12, 2021.
(60) Provisional application No. 63/256,110, filed on Oct. 15, 2021.

(51) Int. Cl. C07C 29/82; B01D 3/36; B01D 3/14

(52) U.S. Cl. CPC C07C 29/82; B01D 3/36; B01D 3/148

(57) ABSTRACT

The present disclosure provides for organic solvent production via distillation and dehydration by: directing portions of a feed stream to a first and second distillation columns operating at a different pressures from each other, wherein the organic solvent is preferably an alcohol and more preferably ethanol; generating, in the first distillation column, a vaporous first overhead stream; directing the vaporous first overhead stream directly to a rectification system; generating, in the second distillation column, a vaporous second overhead stream; forming a condensed second overhead stream from the vaporous second overhead stream; directing, at least a portion of the condensed second overhead stream to the rectification system; generating, via the rectification system, a third overhead stream; directing at least a portion of the third overhead stream to a separation system; and generating, in the separation system, an enriched solvent stream.

### CROSS-REFERENCES TO RELATED APPLICATIONS

The present disclosure claims the benefit of and priority to U.S. Provisional Application 63/220,837 filed on Jul. 12, 2021 having the title “DISTILLATION, DEHYDRATION AND EVAPORATION SYSTEMS AND METHODS FOR ORGANIC SOLVENT PRODUCTION”, and U.S. Provisional Application 63/256,110 filed on Oct. 15, 2021 having the title “DISTILLATION AND DEHYDRATION SYSTEMS AND METHODS FOR ORGANIC SOLVENT PRODUCTION”, which are incorporated by reference herein in their entireties.

### BACKGROUND

Some organic solvent production systems, such as ethanol production systems, include molecular sieve units (MSUs) for dehydrating a feed vapor to further separate water and organic solvent mixture beyond the azeotrope. The MSUs typically include two or three beds filled with zeolite pellets, which adsorb water to produce anhydrous vapor until the pellets are saturated with water. While the first bed undergoes a regeneration cycle, the feed vapor coming from the rectifier, or rectifier/stripper, column can be switched to a second bed for continued dehydration. Desorption/depressurization with or without redirecting a portion of freshly dehydrated organic solvent (e.g., alcohol) into the first bed to remove the water from the saturated zeolite beads, forms a regenerate stream (e.g., MSU regen). Due to the water desorption, the regenerate stream has an organic solvent concentration between 50 and 80 vol %, and is recycled to upstream distillation for reprocessing. As such, dehydration with MSUs in typical systems has a number of disadvantages. For example, as a large portion of organic solvent is continuously recycled, (1) capacity in the upstream distillation is used up for reprocessing the MSU Regen, (2) capacity in the MSU itself is used up to essentially dehydrate its own regenerate stream for recycling, and (3) additional energy or steam and cooling water are used for the reprocessing of the MSU Regen.

Some typical organic solvent production systems include membrane dehydration. For example, the MSU Regen may be treated by a membrane dehydration system including a stripper column and a membrane. Such membrane dehydration systems, however, are typically used in conjunction with MSUs.

Therefore, there exists a need for processes and systems that overcome the limitations of typical processes for organic solvent production, and in particular for ethanol production.

### SUMMARY

The present disclosure provides new and innovative organic solvent (e.g., ethanol) production systems and methods that increase capacity and reduce energy consumption as compared to typical organic solvent production systems and methods.

In some aspects, the provided system enables the complete replacement of molecular sieves by membranes and thereby excludes the production of a regeneration stream. Compared to some typical ethanol production systems, the provided system in such aspects may enable a reduction of natural gas consumption of over 4,000 BTU/gal. Additionally, the provided system in such aspects enables additional capacity while fully replacing molecular sieves during dehydration. This is possible for a number of reasons, including one or more of (i) the regen stream is now avoided which allows for additional capacity at distillation, (ii) the installation of a medium pressure column that allows for higher beer stripping capacity, (iii) the ability of the membrane separation system to process lower proof feed, which allows the medium pressure column overheads to be directly processed by such system, which avoids upgrading the existing rectifier and/or side stripper, or combined rectifier/side stripper column, and lowers the steam consumption by being capable of treating lower proof feed. The additional steam savings also comes, in part, from the diverse heat integration that the presently disclosed system provides, such as directing both a retentate stream from membrane dehydration and a medium pressure distillation column overhead to an evaporation system. Both integrations enable the complete exclusion of steam consumption in the evaporators.

Compared to previous system in which regen is treated by a membrane dehydration system, the presently disclosed system provides additional reduction of energy consumption as stated previously. Other advantages of the use of membranes over molecular sieves are smaller footprints, easier maintenance, the removal of constant regeneration cycles, and enabling a modular system that allows for expansion by the addition of additional membranes.

In other aspects, the provided system may include only MSU dehydration and not membrane dehydration. In such other aspects, the presently disclosed system provides improved heat integration as compared to typical ethanol production systems including MSU dehydration.

Additional features and advantages of the disclosed method and apparatus are described in, and will be apparent from, the following Detailed Description and the Figures. The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### BRIEF DESCRIPTION OF THE DRAWINGS

FIG. 1 illustrates an example solvent production plant, according to aspects of the present disclosure.

FIG. 2 illustrates a solvent production plant with a separation system including a stripper column and a membrane, according to an aspect of the present disclosure.

FIG. 3 illustrates a solvent production plant with a separation system including a single rectifier/stripper column, according to an aspect of the present disclosure.

FIG. 4 illustrates a solvent production plant with a separation system including a stripper column and a molecular sieve unit, according to an aspect of the present disclosure.

FIG. 5 illustrates a solvent production plant with a separation system including a vaporizer and a membrane, according to an aspect of the present disclosure.

FIG. 6 illustrates a solvent production plant with a separation system including a vaporizer and a molecular sieve unit, according to an aspect of the present disclosure.

FIG. 7 illustrates a solvent production plant including both MSU dehydration and a membrane dehydration system, according to an aspect of the present disclosure.

FIG. 8 illustrates a solvent production plant with a separation system including a stripper column and a membrane, according to an aspect of the present disclosure.

FIG. 9 illustrates a solvent production plant including both MSU dehydration and a membrane dehydration system, according to an aspect of the present disclosure.

FIG. 10 illustrates a solvent production plant with a separation system including a vaporizer and a molecular sieve unit, according to an aspect of the present disclosure.

FIG. 11 illustrates a solvent production plant with a separation system including a stripper column and a molecular sieve unit, according to an aspect of the present disclosure.

FIG. 12 illustrates a solvent production plant with a separation system including a vaporizer and a membrane, according to an aspect of the present disclosure.

FIG. 13 illustrates a solvent production plant with a rectifier column directly connected to a stripper column, according to an aspect of the present disclosure.

FIG. 14 illustrates a solvent production plant with a heat recovery vessel, according to an aspect of the present disclosure.

FIG. 15 illustrates simulation graphs respectively showing a relationship between reflux flow and steam consumption, and between rectifier overhead proof and steam consumption, according to an aspect of the present disclosure.

FIG. 16 is a flowchart of a method for operating a solvent production plant, according to aspects of the present disclosure.

### DETAILED DESCRIPTION

The provided distillation and dehydration system is configured to produce an anhydrous organic solvent (e.g., >99% vol). In the present disclosure, various examples refer to the solvent, which may be understood to refer to any organic solvent, although preferably an alcohol, and more preferably ethanol. Ethanol, however, is merely one example and the following description applies equally to producing another suitable organic solvent using the provided systems and methods. Therefore, any reference to a solvent provided herein may be understood to refer to any suitable organic solvents including: ethanol, methanol, isobutanol, isopropanol, ketones, or the like.

Various purities of the organic solvent may be produced at different purity levels of the example production system. As used herein with respect to the examples given for ethanol, 190 proof (190P) and 200 proof (200P) are used for two purity levels for approximately at least 95% ethanol by volume and at least 99% ethanol by volume, respectively, but other purity levels may be specified for use according to the present disclosure.

Additionally, various materials may be referred to herein as “freed” of another material (e.g., solids-freed, solvent-freed, water-freed), indicating that the first material has been distilled, filtered, or otherwise separated to remove (or be freed of) at least a portion of the second material. For example, a base liquid containing fifty percent water and fifty percent of an organic solvent (e.g., ethanol) may be subject to a first distillation process to produce a first water-freed stream of thirty percent water and seventy percent of the organic solvent, which may be subject to a second distillation process to produce a second water freed-stream of ten percent water and ninety percent of the organic solvent. In contrast, various materials may be referred to herein as “enriched” with another material (e.g., solvent enriched), indicating that the first material has been distilled, filtered, concentrated, or otherwise supplemented to increase a concentration of the second material. Using the previous examples, the water-freed streams may also be considered to be solvent enriched streams, and the remaining base material (from which the solvent enriched streams were separated) may be considered to be water enriched streams in comparison to their respective inputs.

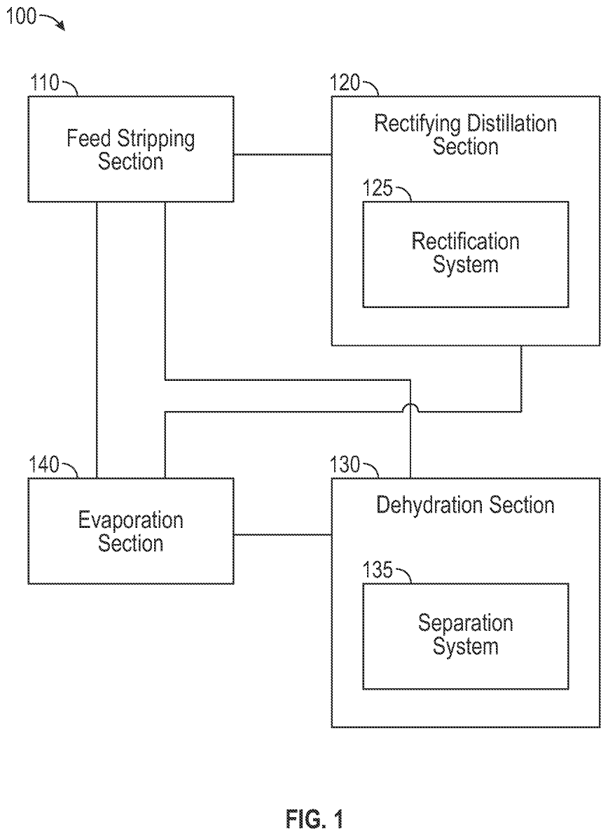

FIG. 1 illustrates an example solvent production plant 100 for an organic solvent, such as an alcohol, according to aspects of the present disclosure. In at least some aspects, the provided solvent production plant 100 can be described as including four sections: a feed stripping section 110, a rectifying distillation section 120, a dehydration section 130, and an evaporation section 140.

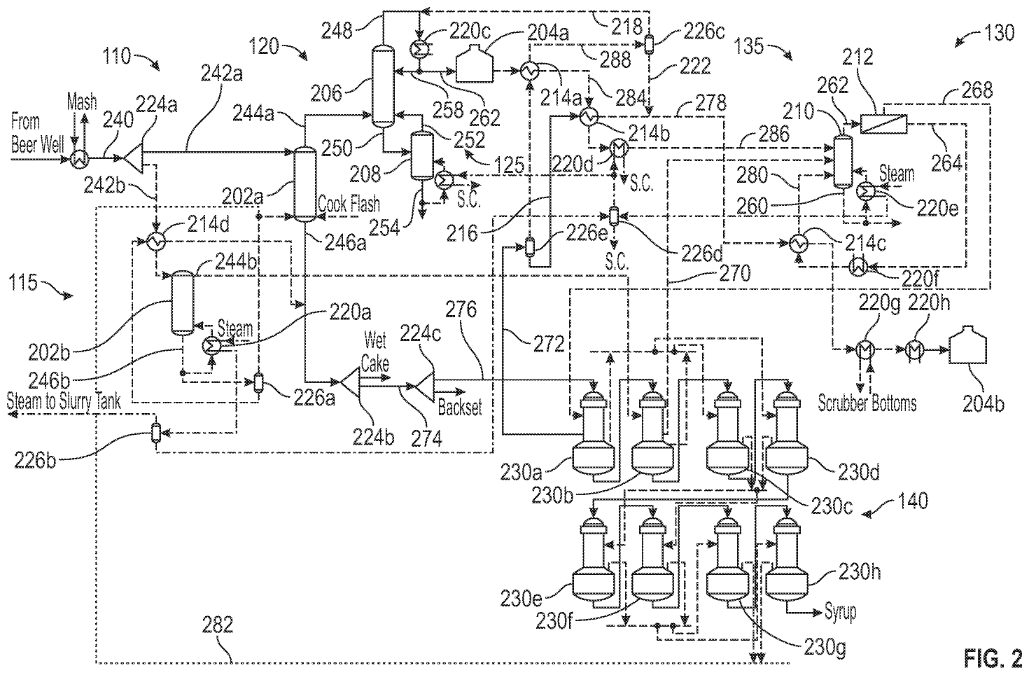

The feed stripping section 110 includes the two distillation columns (that operate at different pressures from one another) and may include various heat exchangers, splitters, flash vessels or the like arranged as in any of FIGS. 2-14. The distillation columns receive portions of a feed stream to produce respective overhead stream and bottom streams to distill the organic solvent from the feed stream.

The rectifying distillation section 120 includes a rectification system 125, which may include one of a rectifier column in direct fluid communication with a vaporizer or stripper column in the dehydration section via a bottom stream from the rectifier column, a rectifier column in direct fluid communication (via a bottom stream) with a side stripper included in the rectification system 125, or an integrated rectifier column and side stripper. Additionally, the rectifying distillation section 120 includes various heat exchangers, splitters, flash vessels, and storage tanks, which may be arranged as illustrated in any of FIGS. 2-14.

The dehydration section 130 includes a separation system 135, which may include various combinations of membranes, molecular sieve units (MSU), stripper columns, and vaporizers to remove water from a rectified organic solvent stream received from (at least) the rectifying distillation section 120 and produce an anhydrous organic solvent stream (e.g., a stream with a higher concentration by volume of the organic solvent due to the further removal of water from the rectified organic solvent stream). Additionally, the dehydration section 130 includes various heat exchangers, splitters, flash vessels, and storage tanks, which may be arranged as illustrated in any of FIGS. 2-14.

The evaporation section 140 includes one or more evaporators that provide for the transfer of heat energy from various “hot” streams of material in the system to various “cold” streams or the environment (e.g., heat venting). In various aspects, a working fluid (e.g., water) in the evaporators extracts thermal energy from a “hot” stream, and provides that thermal energy (e.g., via steam) to another stream (e.g., the “cold” stream) in a heat exchanger. Additionally, the evaporation section 140 includes various heat exchangers, splitters, flash vessels, and storage tanks, which may be arranged as illustrated in any of FIGS. 2-14.

Various components of the presently disclosed systems may be in fluid communication with one another, such as through piping. Two components in fluid communication with one another may be in direct fluid communication (e.g., piping directly connects the two components) or may have intermediate components or processing between the two components, such as filters, pumps, heaters, odor removal vessels, etc.

FIG. 2 illustrates a detailed layout of an example organic solvent production system, according to aspects of the present disclosure in accordance with FIG. 1. Each of the FIGS. 2-14 illustrate detailed layouts for example organic solvent production system in accordance with FIG. 1. Accordingly, the layout for each of the sections 110-140 may be taken from one or more of the detailed layouts, and the detailed layouts for the different sections 110-140 may be provided in different Figures. Stated differently, one of ordinary skill in the art may select a design for a first section from a first one of FIGS. 2-14 and a design for a second section from a second one of FIGS. 2-14. Additionally, one of ordinary skill in the art will appreciate that the detailed layouts may use additional routing features, flow meters, filters, valves, insulation, pumps, or the like that will vary across different deployment environments, and the inclusion of such features (and other minor elements) may be done without undue experimentation when applying the present disclosure. The discussion of features described in relation to one of FIGS. 2-14 may therefore be applied to the common or shared elements in the other detailed layouts.

As shown in the detailed layout of FIG. 2, in the feed stripping section 110, a first splitter 224a (generally or collectively, splitter 224) splits a feed stream 240 (e.g., beer) comprising of a mixture of ethanol (or other suitable organic solvent), water, and solids into two portions 242a-b (generally or collectively, portion 242). The first portion 242a is directed to a first distillation column 202a, (e.g., a beer column (BC)), which thereby forms a solid-freed vaporous overhead stream 244a and a solvent-freed bottom stream 146a. The second portion 242b is directed to a second distillation column 202b, which thereby forms a solid-freed vaporous overhead stream 244b and a solvent-freed bottom stream 246b. In some aspects, the first distillation column 202a operates at a different pressure than the second distillation column 202b, and the relative pressures may include the first distillation column 202a operating at a higher or a lower pressure than the second distillation column 202b.

In some aspects, the first distillation column 202a is driven by process vapors through direct injection, such as vapors from an evaporator 230a-h (generally or collectively, evaporator 230) in the evaporation section 140. In some aspects, the first distillation column 202a is driven by vapors from process streams generated in flash vessels 226a-z (generally or collectively, flash vessels 226). In some aspects, the first distillation column 202a is driven by cook flash vapors. For instance, in the example illustrated in FIG. 2, the first distillation column 202a is driven by a combination of fourth effect vapors, cook flash and vapors generated from flashing a portion of the solvent-freed bottom stream 246b from the second distillation column 202b or other process streams. In other aspects, the first distillation column 202a may additionally or alternatively be driven by a distillation column reboiler (not illustrated in FIG. 2) with a combination of either evaporator vapors, cook flash, vapors generated from flashing a portion of the solvent-freed bottom stream 246b from the second distillation column 202b, or other process streams.

In some aspects, the second distillation column 202b is driven by process vapors through direct injection. In other aspects, the second distillation column 202b may additionally or alternatively be driven by steam through a distillation column reboiler (e.g., heat exchanger 220a (generally or collectively, heat exchanger 220). For example, in the illustrated aspect, the second distillation column 202b is driven only by a distillation column reboiler 220a. In some instances, steam condensate from the distillation column reboiler 220a is flashed in a flash vessel 226a (generally or collectively, flash vessel 226). In such instances, the low pressure steam generated by the flash vessel 226a may be used to drive the reboiler 226b of the side stripper 208 in the rectifying distillation section 120 and/or heat an overhead stream 248 of the rectifier column 206, as illustrated, or may be used to heat any other suitable stream having a lower temperature.

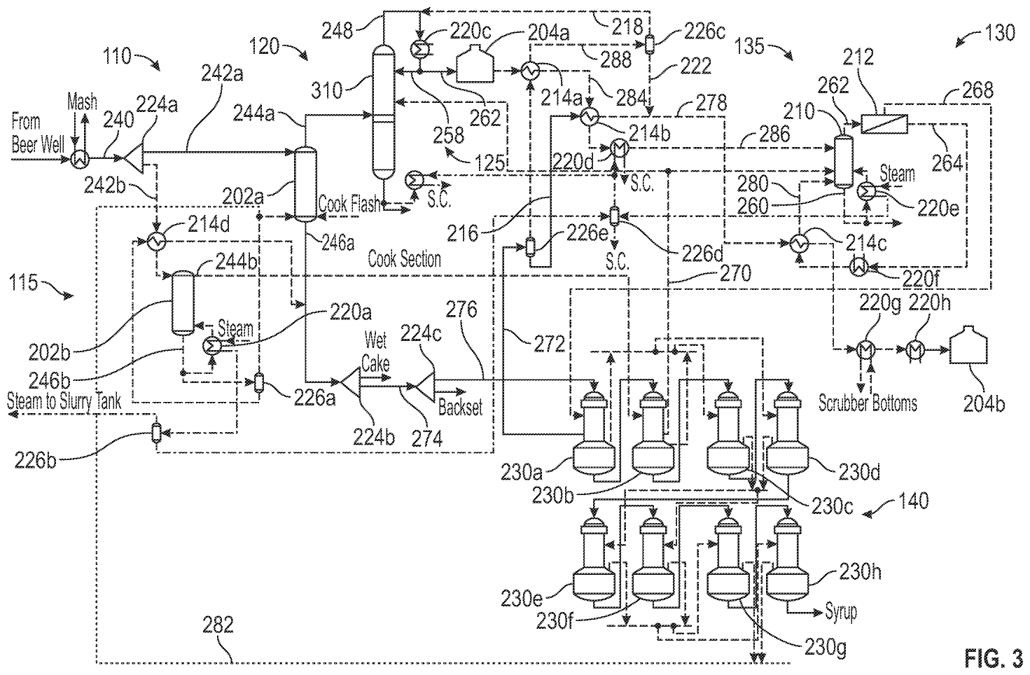

The vaporous overhead stream 244a of the first distillation column 202a may be directed straight (e.g., without any intervening components other than piping) to a rectifier column 206 of the rectifying distillation section 120. Stated differently, the vaporous overhead stream 244a of the first distillation column 202a may be introduced into the rectifier column 206 as a vapor without first being condensed. The vaporous overhead stream 244b of the second distillation column 202b may be condensed. In the example system of FIG. 2, the vaporous overhead stream 244b of the second distillation column 202b is directed to a plurality of evaporators 230 where the vaporous overhead stream 244b is condensed such that it transfers latent energy thereby generating vegetal steam (evaporator vapor) and results in a condensed second overhead stream 270. In some aspects, the condensed second overhead stream 270 of the second distillation column 202b is directed to a separation system 135 of the dehydration section 130 from at least one of the evaporators 230. In other aspects, the condensed second overhead stream 270 of the second distillation column 102b is directed to a rectifier column 206 of the rectifying distillation section 120 from at least one of the evaporators 230. In other aspects still, a first portion of the condensed second overhead stream 270 of the second distillation column 202b is directed to the separation system 135 from at least one of the evaporators 230 while a second portion of the condensed second overhead stream 270 of the second distillation column 202b is directed to the rectifier column 206 from at least one of the evaporators 230. For example, FIG. 3, illustrates a detailed layout of a solvent plant according to FIG. 1 that includes a split pipe from one of the evaporators 230 enabling one stream to be directed to the separation system 235 and a second stream to be directed to the rectifier column 206.

In some aspects, the bottom stream 246b of the first distillation column 202a is directed to the evaporation section 140. In some aspects, at least a portion of the solvent-freed bottom stream 246b of the second distillation column 202b is directed to the evaporation section 140. For instance, in the illustrated example of FIG. 2, the bottom streams 146a, 146b of both the first distillation column 202a and the second distillation column 202b are directed to the evaporation section 140. In some instances, a portion of the bottom stream 146b of the second distillation column 202b is directed to the evaporation section 140 via a flash vessel 226 and/or a heat exchanger 220 to recover at least a portion of the sensible heat of the bottom stream 246. In some aspects, a portion of the bottom stream 246b of the second distillation column 202b is directed to the first distillation column 202a. In some aspects, a portion of the bottom stream 246b of the second distillation column 202b is directed to a flash vessel 126a where the vapors generated are directed with the evaporator vapors 282 to drive the first distillation column 202a or rectifier column 206. In some aspects, a remaining liquid portion resulting from flashing the portion of the bottom stream 246b of the second distillation column 202b exchanges heat with another process stream prior to being directed to the evaporation section 140.

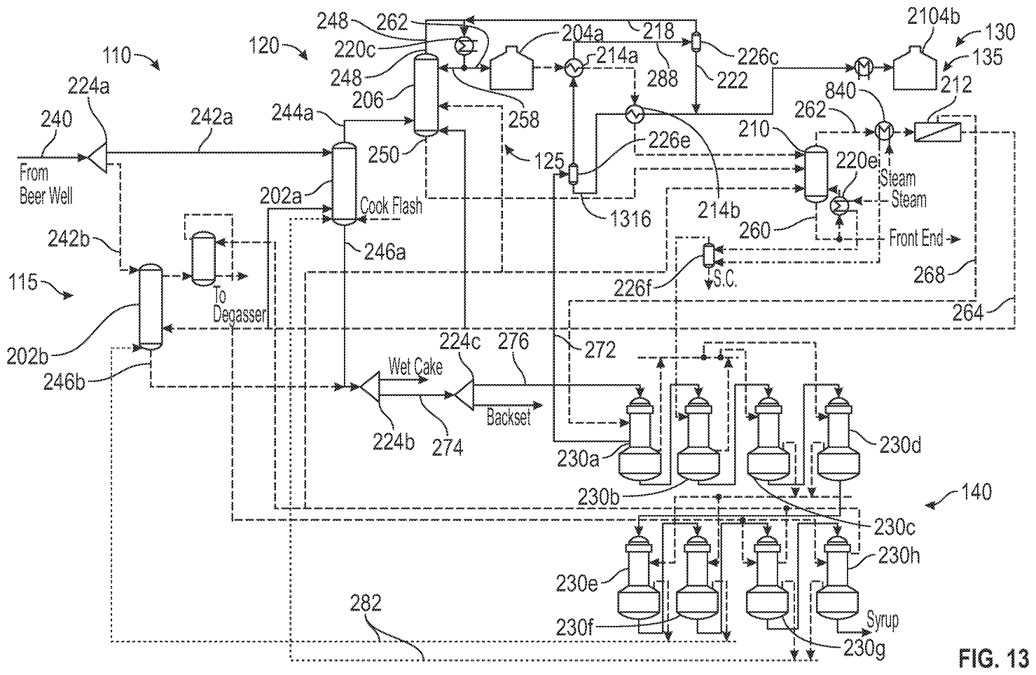

In at least some aspects, the rectifying distillation section 120 may include a rectifier column 206 and a side stripper 208 (as in FIG. 2), a combined rectifier/stripper column 310 (as in FIG. 3), or a rectifier column 206 that omits the side stripper 208 (as in FIG. 13) and places the rectifier column 206 in fluid communication with a stripper column 210 in the dehydration section 130. The rectifier/stripper column 310 is a distillation unit in which both rectification and stripping happens. In some aspects, the rectification system 125 includes a rectifier column 206 in fluid communication with a separate side stripper 208 (as in FIG. 2). While various examples, show the rectification system 125 that includes a separate rectifier column 206 and side stripper 208, it should be appreciated that the following description applies equally to a single rectifier/stripper column 310 or an architecture that omits a side stripper 208 in the rectifying distillation section 120. For instance, streams described as being directed to or from the rectifier column 206 or to the side stripper 208 may be directed to or from a single rectifier/stripper column 310. As stated above, the vaporous overhead stream 244a from the first distillation column 202a may be directed straight to the rectifier column 206, which thereby forms a solvent-rich overhead stream 248 and a bottom stream 250. In various aspects, the solvent-rich overhead stream 248 formed by the rectifier column 206 may be ethanol at any concentration below the Azeotropic concentration. In one example, a solvent-rich overhead stream 248 formed by the rectifier column 206 is 190-proof (190P). The rectifier bottom stream 250 is directed to the side stripper 208 in some aspects, which may thereby form an overhead stream 252 directed back to the rectifier column 206 and a solvent-freed bottom stream 254. In some aspects, the solvent-rich overhead stream 248 is condensed into a condensed solvent-rich overhead stream 256. In some aspects, a portion of the condensed solvent-rich overhead stream 256 is stored in a storage tank 204a (generally or collectively, storage tank 204). In some aspects, a portion of the condensed solvent-rich overhead stream 256 is returned to the rectifying distillation section 120 as a reflux stream 258. In various aspects, at least a portion of the condensed solvent-rich overhead stream 256 is directed to a separation system 135 of the dehydration section 130.

In various aspects, the solvent-freed bottom stream 254 is directed to another area of the solvent production plant (e.g., the cook section) in which the provided system is located. In some aspects, the side stripper 208 is driven by direct vapor injection and/or steam. In other aspects, the side stripper 208 is driven by process vapors or steam via a reboiler 220b. In some examples, a first portion of the solvent-freed bottom stream 254 generated by the side stripper 208 is directed to a reboiler 220b driven by either steam or process flash vapors and a second portion of the solvent-freed bottom stream 254 is forwarded to a front end of the solvent production plant 100 in which the provided system is located.

In at least some aspects, the dehydration section 130 includes a separation system 135. In the example system of FIG. 2, the separation system 135 includes a stripper column 210 and a membrane 212 (e.g., a semi-permeable membrane). The stripper column 210 generates an overhead vapor stream from a solvent-water concentrated feed stream that is directed to contact the membrane 212. The stripper column 210 may also generate a bottom stream 260 that may be directed to another area of the solvent production plant 100 in which the provided system is located. In various aspects, the bottom stream 260 from the stripper column 210 may be used to heat a suitable cold stream (e.g., steam condensate, process water, scrubber water, 190P, a regenerate stream, a feed stream 240, etc.) to recover heat that would otherwise be wasted. In some aspects, the stripper column 210 may be driven by a reboiler 220e. In some examples, steam condensate (SC) from the reboiler 220e is flashed in a flash vessel 226d. In such examples, the low pressure steam generated by the flash vessel 226d may be used to drive the reboiler 220b of the side stripper 208 in the rectifying distillation section 120 or heat the overhead stream 248 of the rectifier column 206, as illustrated, or may be used to heat any other suitable stream having a lower temperature.

The membrane 212 continuously removes water from the solvent-water concentrated feed vapor stream 262 to produce a vaporous water-rich stream (a permeate stream 264) and a vaporous anhydrous solvent-rich stream (a vaporous retentate stream 268). For example, a anhydrous solvent-rich vaporous retentate stream 268 may include 99% by volume or higher of ethanol. In some aspects, the membrane 212 is a polymer membrane, which may be built on hollow fibers. In various aspects, a selective layer of the membrane 212 is placed on either the outside (e.g., shell side) or the inside (e.g., lumen side) of the hollow fibers. In other examples, the membrane 212 takes other suitable forms that suitably dehydrate a feed vapor stream 262 as part of a high-grade solvent production process, such as tubular membranes including zeolites membranes or spiral wound membranes.

In at least some aspects, the vaporous retentate stream 268 generated by the membrane 212 is directed to at least one evaporator 230 in the evaporation section 140. In such aspects, the vaporous retentate stream 268 is condensed in the at least one evaporator 230 into a liquid retentate stream 278, which may be directed from the at least one evaporator 230 in the evaporation section 140 to an economizer 214b (generally or collectively economizer 214 or condenser 214) in the rectifying distillation section 120. In some aspects, the liquid retentate stream 278 from the evaporators 230 is directed to a flash vessel 226d where the produced 200-proof flash vapor stream 216 can recover its heat elsewhere and be directed to a CO₂ removal system. In some aspects, the CO₂ removal system is a low-pressure flash vessel 226c in which a vapor stream 218 and a liquid stream 222 are generated. The vapor stream 218 is directed to a 190-proof heat exchanger 220c and the liquid stream 222 is directed into the liquid retentate stream 278. In some instances of the provided system, the liquid retentate stream 278 from the 200P flash vessel 226e is directed to an economizer 214b. Thermal energy may be further recovered from the liquid stream 222 against other process streams (e.g., permeate stream 264, scrubber bottoms streams). For example, the liquid retentate stream 278 illustrated in FIG. 2 is cooled further by heating both the scrubber bottoms in one heat exchanger 220g and the liquid permeate stream 264 in another heat exchanger 220h. In at least some aspects, the cooled liquid retentate stream 278 may be directed to a tank 204b (e.g., 200P tank) for storage.

In various examples, the vaporous permeate stream 264 generated by the membrane 212 is condensed. For instance, the heat available in the vaporous permeate stream 264 may be used to heat a suitable cold stream (e.g., steam condensate, process water, scrubber water, 190P, a regenerate stream, a feed stream, etc.) at a condenser 214c, thereby condensing the vaporous permeate stream 264 into a liquid permeate stream 280.

In some examples, such as illustrated in FIG. 2, the condensed liquid permeate stream 280 is directed back to the stripper column 210. The liquid permeate stream 280 may be heated by a suitable hot stream (e.g., flash vapors, side stripper bottom stream, stripper column bottom stream, retentate liquid, etc.) in a heat exchanger 220f prior to being introduced into the stripper column 210 in some aspects. For instance, in the example of FIG. 2, the liquid permeate stream 280 is heated by the liquid retentate stream 278 in a heat exchanger 220.

In various aspects, the evaporation section 140 includes an evaporation system of one or more evaporators 230. In some aspects, vapors generated from a first effect evaporator 230a may be used to drive a second effect evaporator 230b. In some aspects, vapors generated from the second effect evaporator 230b may be used to drive a third effect evaporator 230c. In various aspects, the number of evaporation steps varies from two to eight (e.g., using a fourth effect evaporator 230d, fifth effect evaporator 230e, etc.). In various aspects, one or more of n effect vapors from n evaporators 230 are used to drive the distillation system. In some examples, fourth effect vapors from a fourth effect evaporator 230d are used to drive the first distillation column 202a.

In the evaporation section 140, the bottom stream 246a of the first distillation column 202a and/or the bottom stream 246b of the second distillation column 202b are subjected to a splitter 224 (e.g., a centrifuge system) in which concentrated solids (wet cake) and a low-solids concentration solution (thin stillage 274) are produced. The thin stillage 274 may then be split into two streams: backset and evaporator feed 276. An advantage of the provided system is that backset and evaporator feed 276 ratios can be adjusted and the recycle of backset to the front-end of the plant can be reduced, which improves plant yields and efficiency. The evaporator feed 276 is subjected to the evaporators 230 to increase the solids concentrations in the evaporator feed 276. In some aspects, the evaporator feed 276 receives overhead streams 244a-b from the distillation columns 202a-b to drive evaporation in at least one evaporator 230. In at least some aspects, a vaporous retentate stream 268 from the separation system 135 in the dehydration section 130 is used to drive the evaporation section 140. In some instances, the vaporous overhead stream 244 from a distillation column (e.g., the overhead stream 244b of the second distillation column 202b) is used to drive the evaporation. One advantage of the provided system is the reduction (or elimination) for steam to used drive the evaporation section 140.

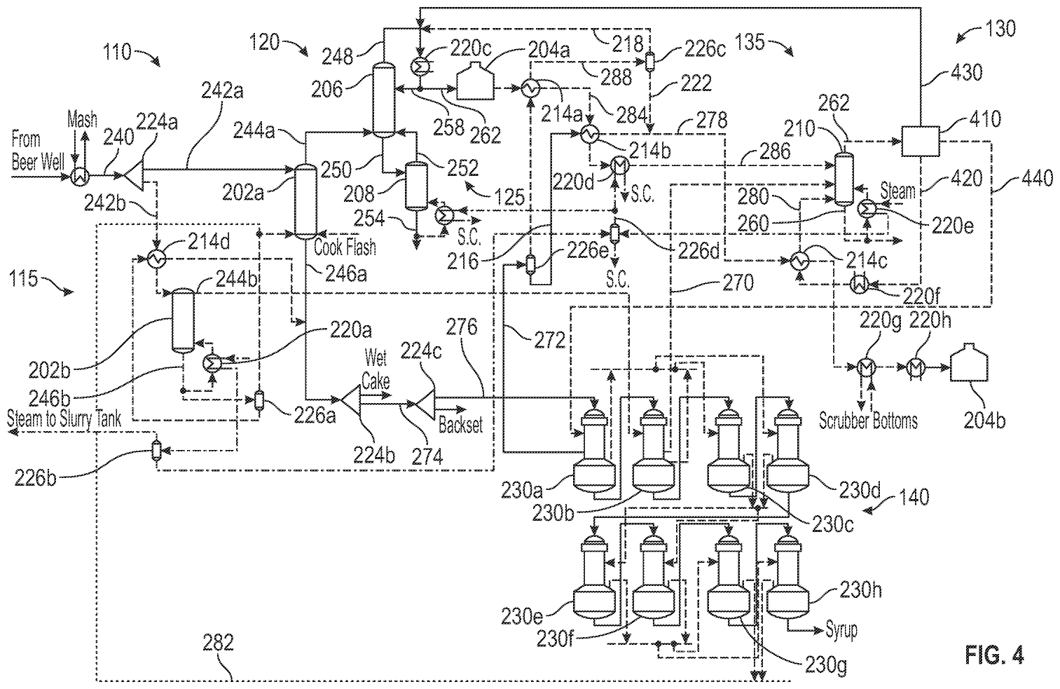

In the example detailed layout of FIG. 4, the separation system 135 includes a stripper column 210 and an MSU 410 including set of molecular sieve beds. The set of molecular sieve beds are configured to generate a product stream 440 (also referred to collectively with the retentate stream 440 as an enriched solvent stream 268/440) and two regenerate streams: a regen stream 420 (e.g., MSU Regen) and a depressure stream 430. The product stream 440 in a solvent production plant 100 is a solvent-rich stream (e.g., 200-proof ethanol). In some aspects, the condensed solvent-rich overhead stream 262 (e.g., 190P) from the rectifier column 206 (and/or via a storage tank 204a) is directed to the stripper column 210, which generates a vaporized stream 262 that is directed to contact the molecular sieve beds of the MSU 410.

The MSU 410 may include multiple beds filled with zeolite pellets, which adsorb water to produce anhydrous vapor until the zeolite pellets are saturated with water. A saturated zeolite pellet bed may be regenerated according to various operator schedules and methodologies. In some instances, freshly dehydrated ethanol may be directed to contact a saturated zeolite pellet bed to remove water from the saturated zeolite pellet bed, which produces a regen stream 420. In other instances, the regeneration is done by vacuum, which generates a regen stream 420 and a depressure stream 430. In various aspects, the regen stream 420 may have an ethanol concentration between 50-80 vol % and therefore is recycled to upstream distillation for reprocessing. For example, the regen stream 420 may be directed to the stripper column 210 of the separation system 135. In various aspects, the depressure stream 430 has a concentration above 80 vol % and may also be recycled to upstream distillation for reprocessing. For example, the depressure stream 430 may be directed to the rectifier column 206 and/or the storage tank 204a that stores a portion of the rectifier overhead stream 248. In instances in which the MSU 410 includes multiple zeolite pellet beds, a saturated zeolite pellet bed may be regenerated while an unsaturated zeolite pellet bed is used to dehydrate a vaporized feed stream 262.

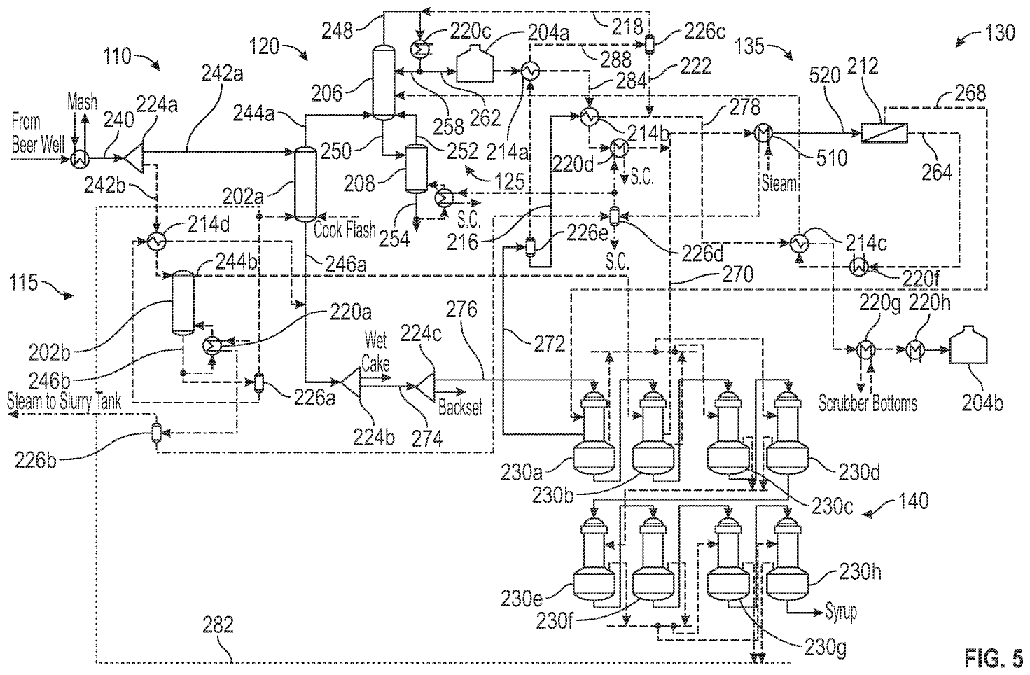

In the example detailed layout of FIG. 5, the separation system includes a vaporizer 510 and a membrane 212. A portion of the solvent-rich overhead stream 248 formed by the rectifier column 206 may be directed to the vaporizer 510. The vaporous overhead stream 244b of the second distillation column 202b is directed to a plurality of evaporators 230 where the vaporous overhead stream 244b transfers latent energy, generating vegetal steam (evaporator vapor 282) and is then directed to the vaporizer 510. The vaporizer 510 generates a vaporized stream 520 that is directed to contact the membrane 212, which thereby forms a vaporous permeate stream 264 and a vaporous retentate stream 268. The vaporous permeate stream 264 may be condensed as described in relation to FIG. 2. In the example of FIG. 5, the condensed liquid permeate stream 280 is directed to the rectifier column 206. In some aspects, the liquid permeate stream 280 is heated by a suitable hot stream (e.g., flash vapors, side stripper bottom stream 254, stripper column bottom stream 260, liquid retentate stream 278, etc.) in a heat exchanger 220 prior to being introduced into the rectifier column 206. For instance, in the example of FIG. 5, the liquid permeate stream 280 is heated by the liquid retentate stream 278 in a heat exchanger 214c.

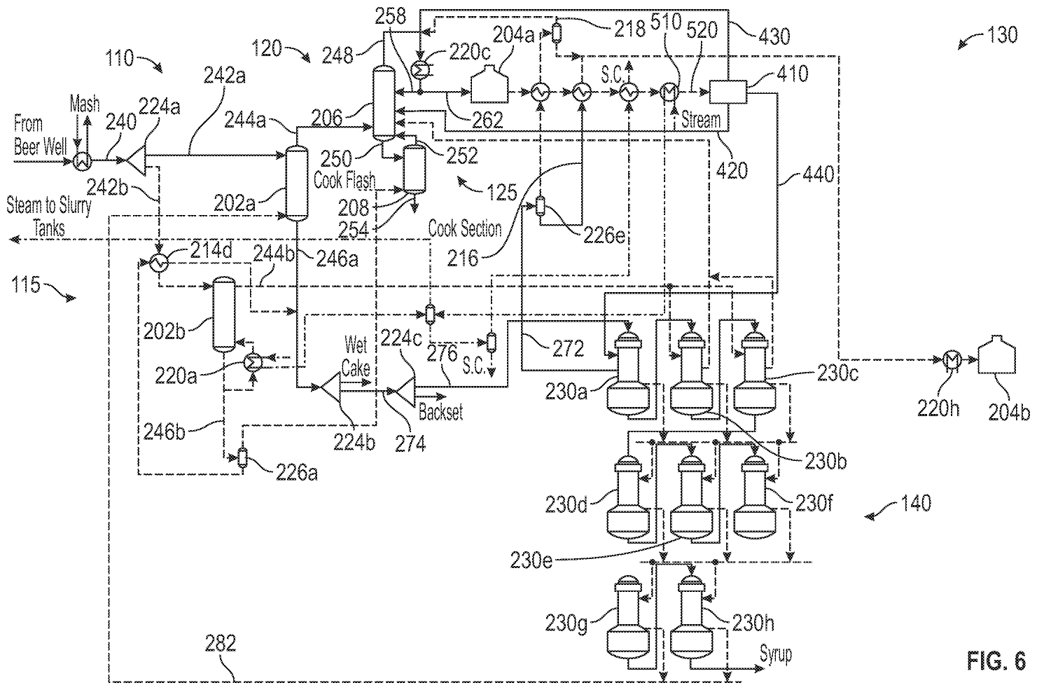

In the example detailed layout of FIG. 6 the separation system may include a vaporizer 510 and an MSU 410 including a set of molecular sieve beds. In at least some examples, the retentate stream 268 (e.g., 200P) of the MSU 410 may be directed to the evaporation section 140 to drive evaporation. In various examples, the overhead stream 244b of the second distillation column 202b may be directed to the rectifier column 206 via at least one of the plurality of evaporators 230. Stated differently, the overhead stream 244b of the second distillation column 202b may be directed to an evaporator 230 in which the overhead stream 244b is condensed and then the condensed second overhead stream 270 is directed to the rectifier column 206. In the illustrated example of FIG. 6, the condensed second overhead stream 270 is directed to the rectifier column 206 that is a separate component in fluid communication with a side stripper 208. In one example, the condensed second overhead stream 270 from the vaporous overhead stream 244b of the second distillation column 202b is 120-proof (120P). In other examples, the condensed second overhead stream 270 from the vaporous overhead stream 244b of the second distillation column 202b may be between 110-proof and 130-proof.

In some aspects, the retentate stream 268 of the MSU 410 may be condensed via the evaporators 230 in the evaporation section 140 and then directed to a flash vessel 126, which thereby forms an overhead vapor stream 218 and a liquid stream 222. In such aspects, the overhead vapor stream 218 may exchange heat with a process stream and the liquid stream may exchange heat in an economizer 214b with a condensed 190P/byproduct stream 284. In some instances, condensed 200P flash vapor 288 is directed to a flash vessel 126c for acidity control by the removal of a vapor portion containing CO₂ that is directed to a 190P heat exchanger 220, which forms a condensed 190P/byproduct stream 222. In such instances, the liquid portion of the acidity control may be directed to the retentate stream 268 of the MSUs 410. In various aspects, the retentate stream 268 of the MSUs 410 may be directed to a tank 204b for storage.

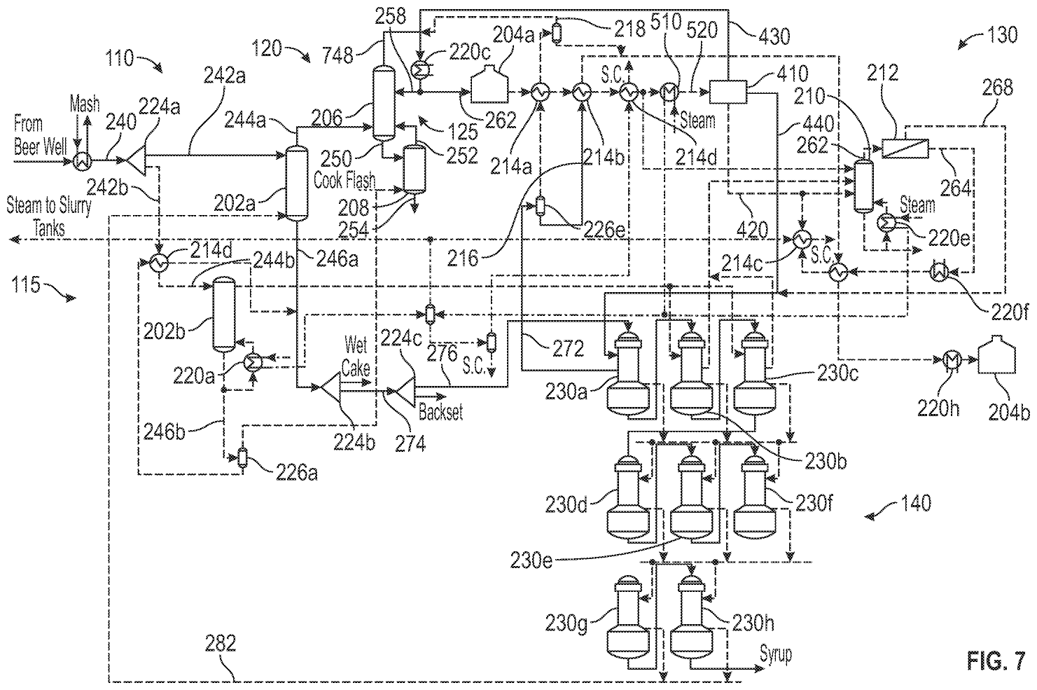

In the example detailed layout of FIG. 7, the dehydration section 130 includes a vaporizer 510, an MSU 410, a stripper column 210, and a membrane 212. In various aspects, the regen stream 420 of the MSU 410 is directed to the stripper column 210.

In various aspects, as described above, the solvent-water concentrated feed stream 286 directed to the stripper column 210 of the separation system 135 includes at least one of: a solvent-rich condensed second overhead stream 284 (e.g., 190P ethanol) from the rectifier column 206 (and/or via a storage tank 204a), condensed second overhead streams 270 from the second distillation column 202b (e.g., 120P ethanol) condensed via at least one of a plurality of evaporators 230, and a permeate stream 264 separated by a membrane 212. In at least some aspects, the solvent-water concentrated feed stream 286 directed to the stripper column 210 of the separation system 135 also includes a liquid permeate stream 280 generated by the membrane 212. Stated differently, the vaporous permeate stream 268 generated by the membrane 212 of the separation system 135 may be condensed and directed as a liquid permeate stream 280 to the stripper column 210 of the separation system 135. Directing a portion of the overhead stream 248 from the rectifier column 206 and condensed second overhead stream 270 (from the second distillation column 202b) to the separation system 135 improves energy efficiency of the process while also improving feed conditions to the membranes 212 and reducing recirculation streams, such as the permeate stream 264. Stated differently, a portion of the rectifier overhead stream 248 (e.g., 190P ethanol) and the overhead streams 248b (e.g., 120P ethanol) from the second distillation column 202b are sent to the dehydration section 130, while taking into account reflux back to the rectifier column 206 and the desired overhead proof from the rectifier column 206 and the stripper column 210 of the separation system 135, and without increasing energy consumption, such that energy efficiency of the process is improved.

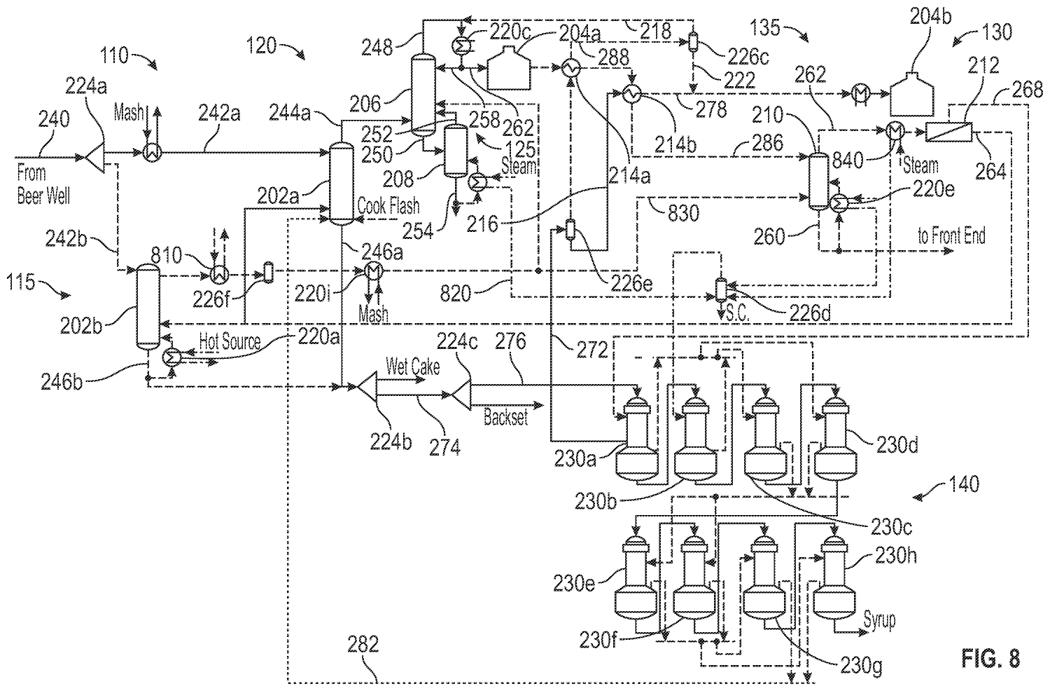

FIG. 8 illustrates a detailed layout of an example organic solvent production system, according to aspects of the present disclosure in accordance with FIG. 1. As described herein, the differences between FIG. 8 and FIG. 2 are provided, with other elements of the detailed layout illustrated in FIG. 8 being substantially similar to those discussed in relation to FIG. 2.

In the feed stripping section 110 for a solvent plant, a splitter 224a splits a feed stream 240 (e.g., beer) comprising of a mixture of an organic solvent (e.g., an alcohol, such as ethanol), water, and solids into two portions 242a, 242b. The first portion 242a is directed to a first distillation column 202a, which thereby forms a solid-freed vaporous overhead stream 244a and a solvent-freed bottom stream 246a. The second portion 242b is directed to a second distillation column 202b, which thereby forms a solid-freed vaporous overhead stream 244b and a solvent-freed bottom stream 246b. In some aspects, the first distillation column 202a operates at a higher pressure than the second distillation column 202b, but the first distillation column 202a may also operate at substantially the same pressure as, or a lower pressure than the pressure that the second distillation column 202b operates at.

In some aspects, the first distillation column 202a is driven by process vapors through direct injection, such as vapors from one or more evaporators 230 in the evaporation section 140. In some aspects, the first distillation column 202a is driven by vapors from process streams generated in flash vessels 226. In some aspects, the first distillation column 202a is driven by cook flash vapors. For instance, in the example illustrated in FIG. 8, the first distillation column 202a is driven by a combination of fourth effect vapors (from the fourth effect evaporator 230d) and cook flash (from a flash vessel 226a). In some aspects, the first distillation column 202a may additionally or alternatively be driven by a distillation column reboiler (not illustrated) with a combination of evaporator vapors, cook flash, flash vapors generated from flashing a portion of the solvent-freed bottom stream 246b from the second distillation column 202b, or other process streams.

In some aspects, the second distillation column 202b is driven by process vapors through direct injection (e.g., a permeate stream 264). In other aspects, the second distillation column 202b is additionally or alternatively driven by steam through a distillation column reboiler 220. For example, in the illustrated aspect, the second distillation column 202b is driven by a distillation column reboiler 220a and direct injection of a permeate stream 264 from a separation system 135.

In some aspects, the vaporous overhead stream 244a of the first distillation column 202a is directed straight (e.g., without any intervening components) to a rectifier column 206 of the rectifying distillation section 120. Stated differently, the vaporous overhead stream 244a of the first distillation column 202a may be introduced into the rectifier column 206 as a vapor without first being condensed. The vaporous overhead stream 244b of the second distillation column 202b, in the example of FIG. 8, is condensed via a heat exchanger 810. In some examples, the condensed second overhead stream 270 from the second distillation column 202b is directed to a storage tank (not illustrated). In some aspects, the condensed second overhead stream 270 from the second distillation column 202b is directed entirely to a separation system 135 of the dehydration section 130 via a first portion of a redirected condensed second overhead stream 830. In other aspects, the condensed second overhead stream 270 from the second distillation column 202b is directed entirely to a rectifier column 206 of the rectifying distillation section 120 via a second portion of the redirected condensed second overhead stream 820. In other aspects still, a first portion of the condensed second overhead stream 270 is directed to the separation system 135 via the first portion of the redirected condensed second overhead stream stream 830 while a second portion of the condensed second overhead stream 270 of the second distillation column 202b may be directed to the rectifier column 206 via a second stream portion of the redirected condensed second overhead stream 820. For example, a first valve (not shown) may be present on the line leading to the separation system 135 and a second valve (not shown) may be present on the line leading to the rectifier column 206. When the first valve is fully open and the second valve is fully closed, the condensed second overhead stream 270 is directed entirely to the separation system 135. When the first valve is fully closed and the second valve is fully open, the condensed second overhead stream 270 is directed entirely to the rectifier column 206. When the first and second valves are each partially open (e.g., half open), a portion of the condensed second overhead stream 270 is directed to each the separation system 135 and the rectifier column 206.

In some examples, the condensed second overhead stream 270 is heated by a suitable hot stream (e.g., beer mash, flash vapors, side stripper bottom stream 254, stripper column bottom stream 260, 200P flash vapor, liquid retentate stream 278, etc.) at a heat exchanger 220i prior to being introduced into the separation system 135 and/or the rectifier column 206.

In various aspects, at least a portion of one or more of the bottom stream 246a of the first distillation column 202a and the solvent-freed bottom stream 246b of the second distillation column 202b may be directed to the evaporation section 140. For instance, in the illustrated example of FIG. 8, the bottom streams 246 of both the first distillation column 202a and the second distillation column 202b are directed to the evaporation section 140.

In at least some aspects, the rectification system 125 of the rectifying distillation section 120 includes a rectifier column 206 in fluid communication with a side stripper 208. In some aspects, the rectifier column 206 and the side stripper 208 may be integrated as a single unit (e.g., a rectifier/stripper column 310). In some aspects, the rectification system 125 may include a rectifier column 206, but omits a side stripper 208. The vaporous overhead stream 244a from the first distillation column 202a may be directed straight to the rectification system 125, which thereby forms a solvent-rich overhead stream 248 and a bottom stream 250. In various aspects, when the solvent-rich overhead stream 248 formed by the rectification system 125 includes ethanol as the solvent, the overhead stream 248 may be between 180-proof and 190-proof. In one example, the solvent-rich overhead stream 248 formed by the rectification system 125 may be 190-proof (190P). The rectifier bottom stream 250 may be directed to the side stripper 208, in various aspects, which may thereby form an overhead stream 252 directed back to the rectifier column 206 and a solvent-freed bottom stream 254. The solvent-rich overhead stream 248 may be condensed. In some aspects, a portion of the solvent-rich condensed overhead stream 262 may be stored in a storage tank 204a (e.g., a 190P tank). In some aspects, a portion of the solvent-rich condensed overhead stream 262 may return to the rectifying distillation section 120 as a reflux stream 258. At least a portion of the solvent-rich condensed overhead stream 262 may be directed to a separation system 135 of the dehydration section 130.

In various aspects, the solvent-freed bottom stream 254 is directed to another area of the solvent production plant (e.g., the cook section) in which the provided system is located. In some aspects, the side stripper 208 is driven by direct vapor injection and/or steam. In other aspects, the side stripper 208 is driven by process vapors or steam via a reboiler 220b. In some examples, a first portion of the solvent-freed bottom stream 254 generated by the side stripper 208 is directed to a reboiler 220b driven by either steam or process flash vapors and a second portion of the solvent-freed bottom stream 254 is forwarded to a front end of the solvent production plant in which the provided system is located. In some examples, steam condensate from the reboiler 220b is flashed in a flash vessel 226. In such examples, the low pressure steam generated by the flash vessel 226 may be used to provide heat to various components of the system. For instance, the low pressure steam may be used to drive an evaporator 230 in the evaporator section 140 or to drive the reboiler 220e of a side stripper 208 in the rectifying distillation section 120, or may be used to heat any suitable stream having a lower temperature than the steam. Steam condensate (S.C.) may be collected in the flash vessel 226 and, in various aspects, returned to a boiler house or Heat Recovery Steam Generator (HRSG) system.

In at least some aspects, the dehydration section 130 includes a separation system 135. In the example system of FIG. 8, the separation system 135 includes a stripper column 210 and a membrane 212 (e.g., a semi-permeable membrane), as further described in relation with FIG. 2. The stripper column 210 generates a vaporous overhead stream 262 from a solvent-water concentrated feed stream 262, and directs the vaporous overhead stream 262 to contact the membrane 212. In at least some aspects, the solvent-water concentrated feed stream 262 includes at least a portion of the solvent-rich condensed overhead stream 262 generated by the rectifier column 206 and the condensed second overhead stream 270 of the second distillation column 202b. In some examples, the vaporous overhead stream 262 generated by the stripper column 210 is heated via steam in a superheater 840. In such examples, steam condensate from the superheater 840 may be flashed in a flash vessel 226. The stripper column 210 may also generate a bottom stream 260 that may be directed to another area of the solvent production plant 100 in which the provided system is located. In some aspects, the stripper column 210 is driven by a reboiler 220e, which may be driven by steam. In some examples, steam condensate from the reboiler 220e for the stripper column 210 may be flashed in a flash vessel 226. In such examples, the low pressure steam generated by the flash vessel 226 may be used to provide heat to various components of the system. For instance, the low pressure steam may be used to drive an evaporator 230 of the evaporation section 140 or to drive the reboiler 220b of the side stripper 208 in the rectifying distillation section 120, or may be used to heat any suitable stream having a lower temperature.

In at least some aspects, the vaporous permeate stream 264 is directly injected (e.g., via direct vapor injection) into the second distillation column 202b. In at least some aspects, a vaporous retentate stream 268 generated by the membrane 212 in the separation system 135 is directed to at least one evaporator 230 in the evaporation section 140. In such aspects, the vaporous retentate stream 268 is condensed in the at least one evaporator 230. A liquid retentate stream 272 may be directed from the at least one evaporator 230 in the evaporation section 140 to an economizer 214b in the rectifying distillation section 120. In some aspects, the liquid retentate stream 272 from the evaporators 230 may be directed to a flash vessel 226e that the latent energy in the produced 200-proof flash vapor can be recovered before being directed to a CO₂ removal system. In various aspects, the CO₂ removal system is a low-pressure flash vessel 226c in which a vapor stream 218 and a liquid stream 222 are generated. The vapor stream 218 may be directed to a 190-proof heat exchanger 220c and the liquid stream may be directed into the liquid retentate stream 278. In some instances of the provided system, the liquid stream 222 from the flash vessel 226e is directed to an economizer 214b. Latent energy in the liquid retentate stream 272 may be further recovered against other process streams (e.g., rectifier overhead stream 248, liquid permeate stream 280, scrubber bottoms). For example, the liquid retentate stream 216 is illustrated in FIG. 8 as heating the overhead stream 262 in an economizer 214b. In at least some aspects, the cooled liquid retentate stream 268 may be directed to a tank 204b (e.g., a 200P tank) for storage.

In various aspects, the evaporation section 140 includes one or more evaporators 230. In some aspects, vapors generated from a first effect evaporator 230a are used to drive a second effect evaporator 230b. In some aspects, vapors generated from the second effect evaporator 230b are used to drive a third effect evaporator 230c. In various aspects, the number of evaporation steps vary from two to eight (e.g., fourth effect evaporator 230d, fifth effect evaporator 230e, etc.). In various aspects, effect vapors from evaporators 230 are used to drive the rectifying distillation section 120. In some examples, fourth effect vapors from a fourth effect evaporator 230d are used to drive the first distillation column 202a.

In the evaporation section 140, the bottom stream 246a of the first distillation column 202a and/or the bottom stream 246b of the second distillation column 202b (e.g., whole stillage) may be subjected to a splitter 224b (e.g., a centrifuge system) in which a stream containing undissolved solids (e.g., wet cake) and a stream containing dissolved solids is produced (e.g., thin stillage 274). The thin stillage 274 is split by a splitter 224c into a backset stream and evaporator feed stream 276. An advantage of the provided system is that backset and evaporator feed ratios can be adjusted and the recycle of backset to the front-end of the plant can be reduced, which improves plant yields and efficiency. For instance, the second distillation column 202b may be driven by a reboiler 220a, thereby reducing water-load to the centrifuge splitter 224b and the evaporator section 140, which allows backset to be reduced. The evaporator feed stream 276 is subjected to the evaporators to increase its dissolved solids concentrations. In some aspects, the evaporator feed stream 276 receives the overhead stream 244b to drive evaporation in at least one evaporator 230. In at least some aspects, the vaporous retentate stream 268 from the separation system 135 in the dehydration section 130 is used to drive the evaporation section 140. In some instances, the vaporous overhead stream 244 from a distillation column 202 (e.g., the overhead stream 244b of the second distillation column 202b) is used to drive the evaporation. One advantage of the provided system is that it reduces (or eliminates) the use of steam to drive the evaporators 230. For instance, heat recovery from the second distillation column overhead stream 244b, 200P vapor (e.g., vaporous retentate stream 268), or flash vapors in the evaporation section 140, in combination with the cascading of energy across the evaporator-effects in the evaporation section 140, helps reduce the use of steam to drive the evaporation section 140.

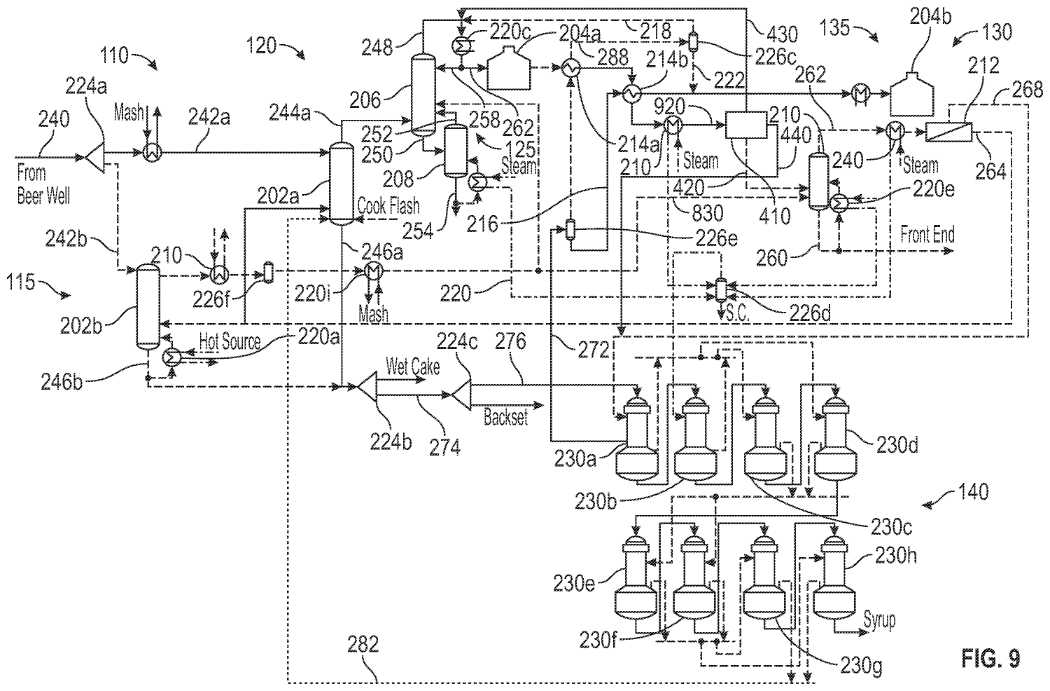

In the example detailed layout of FIG. 9, the separation system 135 of the dehydration section 130 includes a vaporizer and an MSU 410 (including a set of molecular sieve beds) in addition to the stripper column 210 and membrane 212. The MSU 410 is configured to generate a product stream 440 (generally or collectively referred to with the retentate streams 268 as “enriched solvent streams” 268/440) and two regenerate streams as is further described in connection with FIG. 4. The two regenerate streams are a regen stream 420 and a depressure stream 430. The retentate stream 268 is a solvent-rich stream (e.g., 200-proof ethanol). The condensed solvent-rich overhead stream 262 (e.g., 190P) from the rectifier column 206 (and/or via a storage tank 204a) may be directed to the vaporizer 510 which generates a vaporized stream 262 that is directed to contact the MSU 410. In some aspects, the vaporizer 510 is driven by steam. In some examples, steam condensate from the vaporizer 510 is flashed in a flash vessel 226.

The regen stream 420 may have a solvent concentration between 50-80 vol % and therefore is recycled to upstream distillation for reprocessing. For example, the regen stream 420 may be directed to the stripper column 210 of the separation system 135. The depressure stream 430 may have a concentration above 80 vol % of the solvent and may also be recycled to upstream distillation for reprocessing. For example, the depressure stream 430 may be directed to the rectifier column 206 and/or the storage tank 204a that stores a portion of the overhead stream 248 from the rectifier column 206. In various aspects, the product stream 440 is directed to at least one of the evaporators 230 in the evaporation section 140. For example, the product stream 440 may be directed into the vaporous retentate stream 268, which is directed to at least one evaporator 230 in the evaporation section 140.

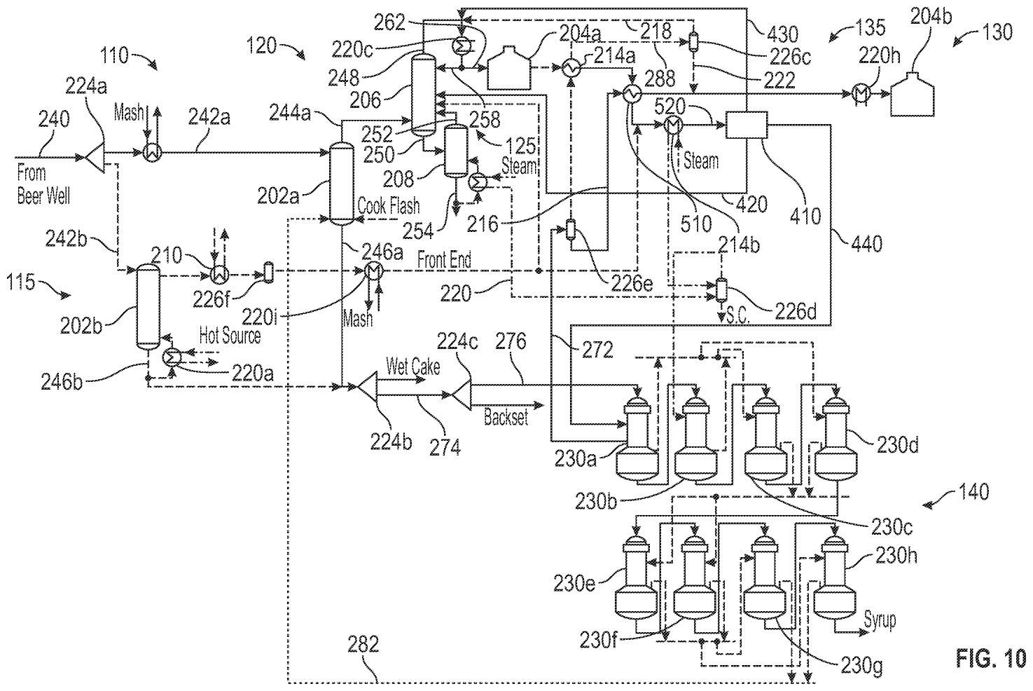

In the example detailed layout of FIG. 10, the separation system 135 of the dehydration section 130 includes a vaporizer 510 and an MSU 410. In this example, the overhead stream 244b of the second distillation column 202b is condensed via a heat exchanger 810. The condensed second overhead stream 270 is then directed to the vaporizer 510. In some aspects, the condensed solvent-rich overhead stream 262 (e.g., 190P ethanol) from the rectifier column 206 (and/or via a storage tank 204a) is also directed to the vaporizer 510. From the condensed second overhead stream 270 of the second distillation column 202b and the condensed solvent-rich overhead stream 262 of the rectifier column 206, the vaporizer 510 generates a vaporized stream 520 that is directed to contact the MSU 410. In the example system of FIG. 10, the regen stream 420 of the MSU is directed to the rectifier column 206.

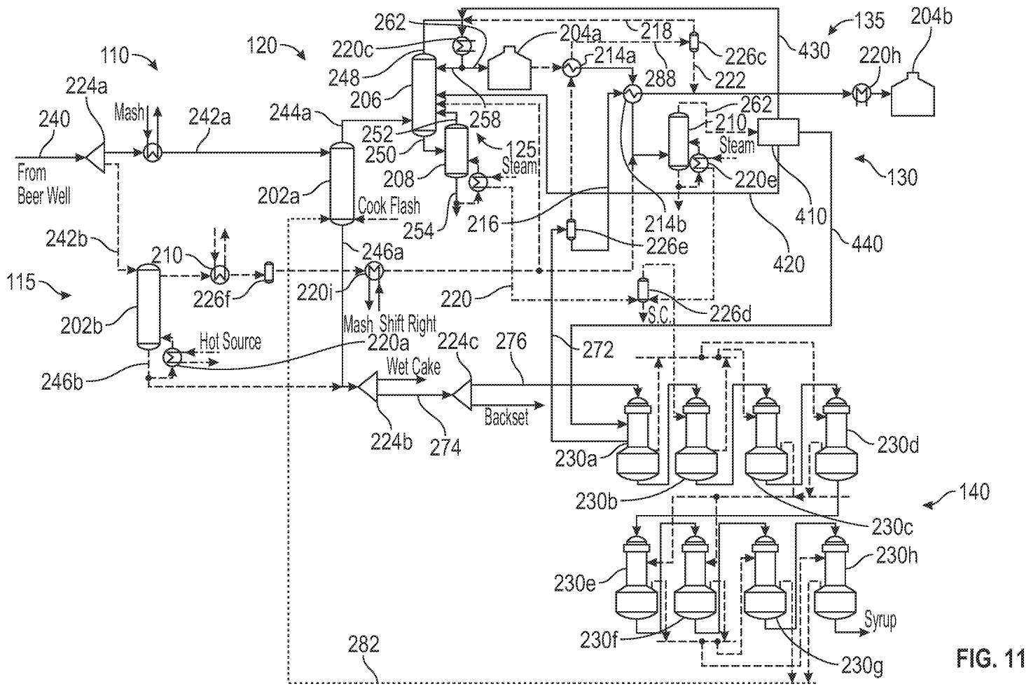

In the example detailed layout of FIG. 11, the separation system of the dehydration section may instead include a stripper column and an MSU. The overhead stream 244b of the second distillation column 202b is condensed via a heat exchanger 810. In some examples, the condensed second overhead stream 270 is directed to the rectifier 208. In some examples, the condensed second overhead stream 270 is directed to the stripper column 210. In some examples, a portion of the condensed second overhead stream 270 is directed to the rectifier 208 and a portion of the condensed second overhead stream 270 is directed to the stripper column 210. In some aspects, the condensed solvent-rich overhead stream 262 (e.g., 190P ethanol) from the rectifier column 206 (and/or via a storage tank 204a) is directed to the stripper column 210. From the condensed second overhead stream 270 of the second distillation column 202b and the condensed solvent-rich overhead stream 262 of the rectifier column 206, the stripper column 210 generates an overhead vapor stream 262 that is directed to contact the MSU 410. In the example system of FIG. 11, the regen stream 420 of the MSU 410 is be directed to the rectifier column 206.

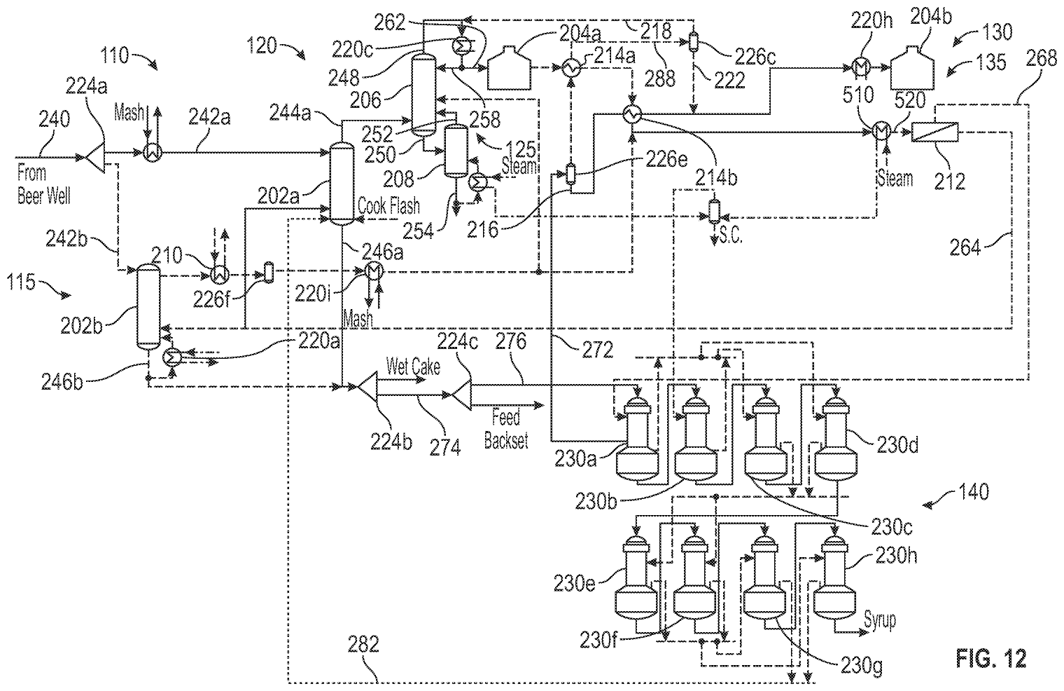

In the example detailed layout of FIG. 12, the separation system 135 of the dehydration section 130 includes a vaporizer 510 and a membrane 212. The overhead stream 244b of the second distillation column 202b is condensed via a heat exchanger 810. The condensed second overhead stream 270 may then be directed to the vaporizer 510. The condensed solvent-rich overhead stream 262 (e.g., 190P ethanol) from the rectifier column (and/or via a storage tank 204a) is also directed to the vaporizer 510. From the condensed second overhead stream 270 of the second distillation column 202b and the condensed solvent-rich overhead stream 262 of the rectifier column, the vaporizer 510 generates a vaporized stream 520 that is directed to contact the membrane 212.

The configuration of the example solvent production systems of FIGS. 8-12 can help provide a number of advantages such as reduced fouling on the second distillation column 202b and reduced configuration changes of an existing solvent production plant's evaporation section 140 for a user to implement the provided solvent production systems of FIGS. 8-12.

In the example detailed layout of FIG. 13, the rectification system 125 that includes a rectifier column 206, but omits a side stripper 208. The rectifier column 206 routes the bottom stream 750 directly to the stripper column 210 in the separation system 130, which in turn may route the bottoms and other waste products to a front end of the solvent production plant 100 for further processing or recycling.

Additionally, FIG. 13 illustrates that the evaporation section 130 may receive steam generated from flashing the bottom stream from the stripper column 210 as well as fresh steam (e.g., from a steam plant).

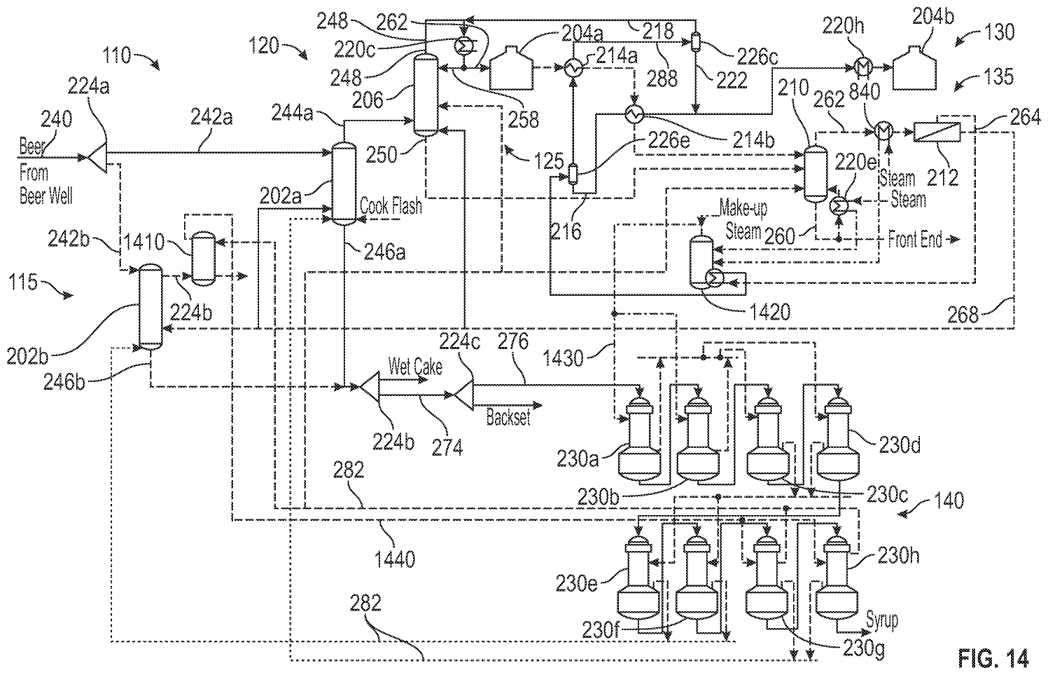

In the example detailed layout of FIG. 14, the feed stripping section 110 includes a liquid-vapor contactor 1410 and the evaporation section 130 includes a steam condensate vessel 1420. The liquid-vapor contactor 1410 is a heat integration vessel that receives the second overhead stream 244b from the second distillation column 202b, and cycles vapors to/from the evaporation section to exchange heat with the overhead stream 244b before it is sent to a degasser for further processing. The liquid-vapor contactor 1410 is designed for counter-current contact of the vaporous second overhead steam 270 with at least a portion of the condensed second overhead stream 270 returning from the evaporators 230 to remove any solids entrained or carried over in the second overhead stream 270. By removing suspended solids that could carry over, the liquid-vapor contactor 1410 reduces the risk of fouling and improves the heat transfer from the vaporous second overhead steam 270 across the evaporators 230. The condensed second distillation column overhead stream 280 in contact with the vaporous second overhead stream 280 in the liquid-vapor contactor 1410 can be sent to various upstream processes (e.g., degassers) for reprocessing before returning to one or both of the first distillation column 202a and the second distillation column 202b. The liquid-vapor contactor 1410 thereby reduces (or prevents) any solids from carrying over to the upstream processes via removal from the bottom streams 246. In various aspects, as part of a centrifuge process, undissolved solids are separated as wet cake and dissolved solids in the thin stillage are concentrated in the evaporators 230 to produce a syrup.

The steam condensate vessel 1420 is another heat integration vessel, which receives steam condensate from the reboiler 220e and the superheater 840. In various aspects, steam condensate from other process areas of the solvent production plant 100 can also be received by the steam condensate vessel 1420 (e.g., from the rectifier-side stripper reboiler or second distillation column reboiler if steam operated). The vaporous retentate stream 268 exchanges latent energy with the steam condensate to generate steam that is mixed with make-up steam to operate various systems and heaters in the solvent production plant 100.

Although illustrated in FIG. 14 in the feed stripping section 110 and the evaporation section 130, in various aspects, various heat integration vessels may be placed throughout the solvent production plant 100 to extract usable heat from one stream and transfer the thermal energy to another (initially colder) stream.

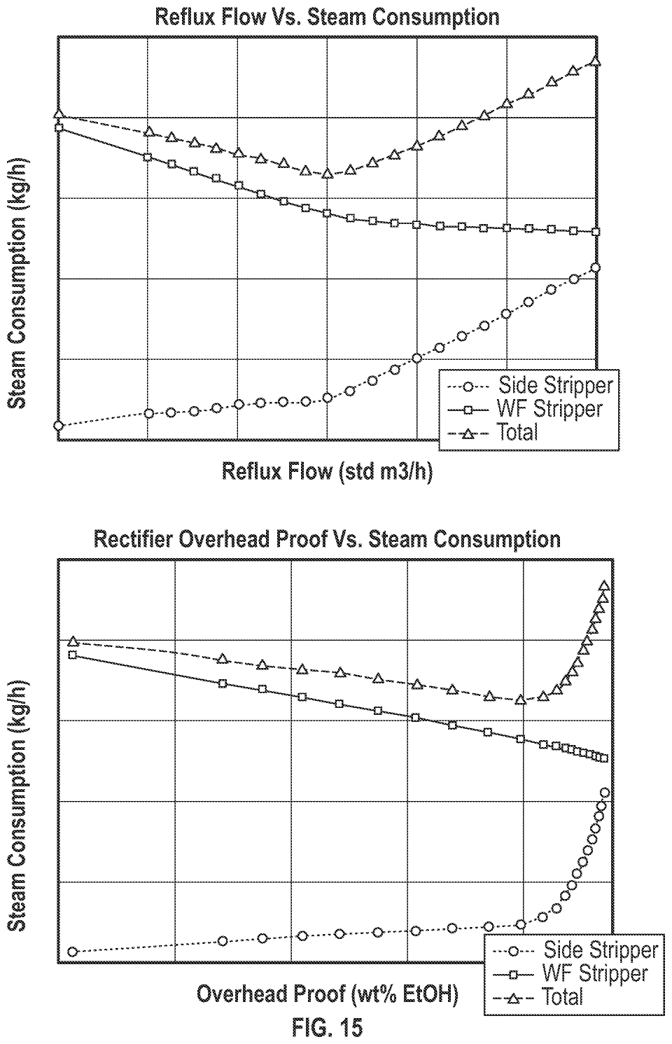

FIG. 15 illustrates graphs showing a relationship between reflux flow and steam consumption, and between rectifier overhead proof and steam consumption, respectively. In various aspects, the provided system may take into account a minimum point of each respective relationship as part of optimizing the energy efficiency of the system.

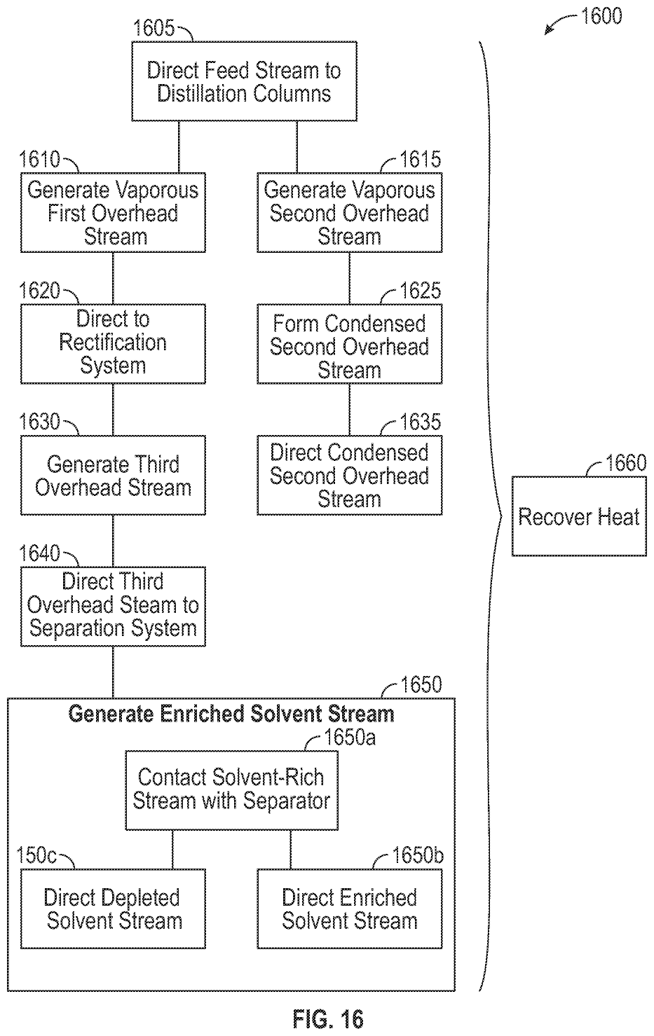

FIG. 16 is a flowchart of a method 1600 for operating a solvent production plant 100, according to aspects of the present disclosure. Method 1600 begins at operation 1605, where a splitter 224a directs a first portion 242a of a feed stream 240 that includes an organic solvent, water, and solids (e.g., an alcohol, such as ethanol, in a beer feed) to a first distillation column 202a, and a second portion 242b of the feed stream 240 to a second distillation column 202b. The first distillation column 202a and the second distillation column 202b operate at a different pressures than each other. In some aspects, the first distillation column 202a operates at a higher pressure than the second distillation column 202b. In some aspects, the first distillation column 202a operates at a lower pressure than the second distillation column 202b.

At operation 1610, the first distillation column 202a generates a vaporous first overhead stream 244a that includes the organic solvent at a higher concentration than in the input streams received by the first distillation column 202a. Additionally, the first distillation column 202a generates a first bottom stream 246a (having a lower concentration of the organic solvent than the input stream), which is removed from the first distillation column 202a to allow for more inputs to be fed into the first distillation column 202a.

At operation 1615, the second distillation column 202b generates a vaporous second overhead stream 244b that includes the organic solvent at a higher concentration than in the input streams received by the second distillation column 202b. Additionally, the second distillation column 202b generates a second bottom stream 246b (having a lower concentration of the organic solvent than the input stream), which is removed from the second distillation column 202b to allow for more inputs to be fed into the second distillation column 202b. In various aspects, the concentration of the organic solvent in the vaporous second overhead stream 244b is different than the concentration of the organic solvent in the vaporous first overhead stream 244a.

In various aspects, one or both of the first distillation column 202a and the second distillation column 202b may receive other inputs in addition to or alternatively to the feed stream 240, which can include cook flash, recycled bottom streams from the distillation column 202, process vapors 282 from the evaporation section 140, a permeate stream (vaporous or condensed) from the dehydration section 130, and combinations thereof.

At operation 1620, the first distillation column 202a directs the vaporous first overhead stream 244a directly to a rectification system 125. As used herein, directing a stream directly from one element of the solvent production plant 100 to another element (e.g., from the first distillation column 202a to a rectifier column 206 or a rectification/stripper column 310) indicates that the stream is routed through pipes with no other intervening elements (e.g., filters, pumps, etc.). Accordingly, the directly routed vaporous first overhead stream 244a leaves the first distillation column 202a as a vapor, and enters the rectifier column 206 or rectification portion of the rectification/stripper column 310 as a vapor.

At operation 1625, the solvent production plant 100 forms a condensed (e.g., liquid) second overhead stream 270 from the vaporous second overhead stream 244b. In various aspects, one or more evaporators 230 in the evaporation section 140 are used to condense the vaporous second overhead stream 244b. In some aspects, a heat exchanger 220 condenses the vaporous second overhead stream 244b while extracting usable heat from the vaporous second overhead stream 244b against a second stream (e.g., a cold stream) of a different, lower initial temperature.

At operation 1630, the rectification system 125 generates a third overhead stream 248 of a solvent-rich overhead stream having a higher concentration of the solvent than the received inputs to the rectification system 125. In various aspects, the third overhead stream 248 is 190P ethanol.

At operation 1635, the solvent production plant 100 directs the condensed second overhead stream 270 for further processing. In some aspects, the solvent production plant 100 directs at least a portion of the condensed second overhead stream 270 to the rectification system 125, to the separation system 135, or both. In aspects in which the rectification system 125 receives, the condensed second overhead stream 270, the second overhead stream 270 is used as an input to generate the third overhead stream (e.g., according to operation 1630).

At operation 1640, the rectification system 125 directs the third overhead stream 248 to a separation system 135 in the dehydration section 130. In various aspects, the rectification system 125 directs the third overhead stream 248 to a storage tank 204a as an intermediate element to store the solvent rich stream before directing the third overhead stream 248 to the dehydration section 130.

At operation 1650, the separation system 145 generates an enriched solvent stream (e.g., a retentate stream 268 or a product stream 440). When the solvent production plant 100 produces an alcohol (e.g., ethanol) as an output, the enriched solvent stream may be 200P alcohol.

In various aspects, generating the enriched solvent stream includes various sub-operations depending on the layout of the solvent production plant 100. For example, at sub-operation 1650a, the solvent production plant 100 contacts a solvent rich-stream (e.g., a solvent-rich overhead stream 262 from a stripper column 210, a vapor stream 510 generated by a vaporizer) with a separator system (e.g., a membrane 212 or an MSU 410), which produces the desired enriched solvent stream (e.g., a retentate stream 268 or a product stream 440) with a high concentration of the solvent and one or more depleted solvent streams (e.g., a permeate stream 264 or a regen stream 420, a depressure stream 430) of remaining material from which the solvent was separated.

In various aspects, after being separated by the separator system, the separator system directs the enriched solvent stream, per sub-operation 1650b, to regenerate one or more beds in an MSU 410, to various cold streams to recover heat from the enriched solvent stream (e.g., per operation 1660) and condense the enriched solvent stream, to an evaporator 230 to condense the enriched solvent stream, to another portion of the separation system 135 (e.g., from an MSU 410 to a stripper column 210 and membrane 212), or to a storage tank 204b for later distribution.

In various aspects, after being separated by the separator system, the separator system directs the depleted solvent stream(s) (e.g., a permeate stream 264 or regen stream 420, a depressure stream 430) back into the solvent production plant 100 for further processing, heat recovery (e.g., per operation 1660), and reprocessing, or out of the production plant 110 for recycling or disposal. In some aspects, sub-operation 1650c includes directing a depressure stream 430 to the rectification system 125 (e.g., the rectifier column 206 or the rectification portion of a rectifier/stripper column 310) and/or the storage tank 204a that stores a portion of the overhead stream 248 from the rectifier column 206. In some aspects, sub-operation 1650c includes directing a depleted solvent stream (e.g., a permeate stream 264 or regen stream 420 to one of the stripper column 210, the vaporizer 510, the rectification system 125 (e.g., the rectifier column 206 or the rectification portion of a rectifier/stripper column 310), and the second distillation column 202b.

At operation 1660, the solvent production plant 100 recovers heat from one of more hot streams of material to one or more cold streams of material. Various heat exchangers in the solvent production plant 100 transfer thermal energy from a first stream of a first temperature to a second stream of a second temperature that is less than the first temperature. The hot streams may be any stream in the solvent production plant 100 with excess heat, and the cold stream may be any stream in the solvent production plant 100 that would otherwise be heated via steam, reboilers, or heating elements using fuel or external energy.

Although illustrated in sequence, the present disclosure contemplates that the various operations described in relation to FIG. 16 may be performed in parallel, as a continuous process, or in different orders than the order shown in FIG. 16. The designation of the operations is therefore provided for the convenience of the reader, and is not intended to specify a preferred order.

The present disclosure can also be understood with reference to the following numbered clauses.

Clause 1: A method (1600), comprising: directing (1605) a first portion (242a) of a feed stream (240) comprising of an organic solvent, water, and solids to a first distillation column (202a) and a second portion (242b) of the feed stream (240) to a second distillation column (202b) operating at a different pressure than the first distillation column (202a), wherein the organic solvent is preferably an alcohol and more preferably ethanol; generating (1610), in the first distillation column (202a), a vaporous first overhead stream (244a); directing (1620) the vaporous first overhead stream (244a) directly to a rectification system (125); generating (1615), in the second distillation column (202b), a vaporous second overhead stream (244b); forming (1625) a condensed second overhead stream (270) from the vaporous second overhead stream (244b); directing (1635), at least a portion of the condensed second overhead stream (270) to the rectification system (125); generating (1630), via the rectification system (125), a third overhead stream (248); directing (1640) at least a portion of the third overhead stream (248) to a separation system (135); and generating (1650), in the separation system (135), an enriched solvent stream (268/440).

Clause 2: The method of any of clauses 1-19, wherein the organic solvent is an alcohol, preferably ethanol.

Clause 3: The method of any of clauses 1-19, wherein the separation system (135) includes a membrane (212) and a vaporizer (510), wherein generating (1650) the enriched solvent stream (268/440) further comprises: contacting (1650a) the membrane (212) with a vapor stream (262) generated by the vaporizer (510), thereby generating a permeate stream (264); and directing (1650c) the permeate stream (264) to the one of the stripper column (210), the vaporizer (510), the rectification system (125), the first distillation column (202a), and the second distillation column (202b).

Clause 4: The method of any of clauses 1-19, wherein the separation system (135) includes a membrane (212) and one of a stripper column (210) or a vaporizer (510), wherein generating (1650) the enriched solvent stream (268/440) further comprises: contacting (1650a) the membrane (212) with a vapor stream (262) generated by the one of the stripper column (210) or the vaporizer (510), thereby generating a retentate stream (268); and directing (1650b) the retentate stream (268) to an evaporator (230) thereby forming a condensed retentate stream (270).

Clause 5: The method of any of clauses 1-19, wherein the separation system (135) includes a stripper column (210) and a membrane (212), wherein generating (1650) the enriched solvent stream (268/440) further comprises: contacting (1650a) the membrane (212) with a vapor stream (262) generated by the stripper column (210), thereby generating a permeate stream (264); and directing (1650c) the permeate stream (264) to the rectification system (125).

Clause 6: The method of any of clauses 1-19, wherein the separation system (135) includes a membrane (212) and one of a stripper column (210) and a vaporizer (510), wherein generating (1650) the enriched solvent stream (268/440) further comprises: contacting (1650a) the membrane (212) with a vapor stream (262/520) generated by the stripper column (210) or the vaporizer (510), thereby generating a retentate stream (268) and a permeate stream (264); directing (1650c) the permeate stream (264) to the second distillation column (202b); and directing (1650b) the retentate stream (268) to an evaporator (230).

Clause 7: The method of any of clauses 1-19, wherein the separation system (135) includes a membrane (212) and one of a stripper column (210) and a vaporizer (510), the method further comprising: contacting (1650a) the membrane (212) with a vapor stream (262/520) generated by the one of the stripper column (210) and the vaporizer (510), thereby generating a permeate stream (264); condensing the permeate stream (264) to form a condensed permeate stream (280); and directing (1650c) the condensed permeate stream (280) to at least one of the stripper column (210), the first distillation column (202a), the second distillation column (202b), and the rectification system (125).

Clause 8: The method of any of clauses 1-19, wherein the separation system (135) includes a molecular sieve unit (410) and one of a stripper column (210) or a vaporizer (510), wherein generating (1650) the enriched solvent stream (268/440) further comprises: contacting (1650a) the molecular sieve unit (410) with a vapor stream (262) generated by the one of the stripper column (210) or the vaporizer (510), thereby generating a regen stream (420); and directing (1650c) the permeate stream (264) to the one of the stripper column (210), the vaporizer (510), the rectification system (125), the first distillation column (202a), and the second distillation column (202b).

Clause 9: The method of any of clauses 1-19, wherein the separation system (135) includes a molecular sieve unit (410) and one of a stripper column (210) or a vaporizer (510), wherein generating (1650) the enriched solvent stream (268/440) further comprises: contacting (1650a) the molecular sieve unit (410) with a vapor stream (262) generated by the one of the stripper column (210) or the vaporizer (510), thereby generating a product stream (440); and directing (1650b) the product stream (440) to an evaporator (230) thereby forming a condensed product stream (440).

Clause 10: The method of any of clauses 1-19, wherein the separation system (135) includes a stripper column (210) and a molecular sieve unit (410), wherein generating (1650) the enriched solvent stream (268/440) further comprises: contacting (1650a) the molecular sieve unit (410) with a vapor stream (262) generated by the stripper column (210), thereby generating a regen stream (20); and directing (1650c) the regen stream (420) to the rectification system (125).

Clause 11: The method of any of clauses 1-19, wherein the separation system (135) includes a molecular sieve unit (410) and one of a stripper column (210) and a vaporizer (510), the method further comprising: contacting (1650a) the molecular sieve unit (410) with a vapor stream (262/520) generated by the one of the stripper column (210) and the vaporizer (510), thereby generating a regen stream (420); condensing the regen stream (420) to form a condensed regen stream (420); and directing (1650c) the condensed regen stream (420) to at least one of the stripper column (210) and the rectification system (125).

Clause 12: The method of any of clauses 1-19, wherein the separation system (135) includes: a membrane (212); a stripper column (210); a vaporizer (510); and a molecular sieve unit (410), the method further comprising: contacting (1650a) the molecular sieve unit (410) with a vapor stream (520) generated by the vaporizer (510), thereby generating a regen stream (420); directing (1650b) the regen stream (420) from the molecular sieve unit (410) to the stripper column (210) to generate a solvent-enriched overhead stream (262); contacting (1650a) the membrane (212) with the solvent-enriched overhead stream (262), thereby generating a retentate stream (268) having a higher concentration of the organic solvent than the solvent-enriched overhead stream (262).

Clause 13: The method of any of clauses 1-19, wherein forming the condensed second overhead stream (270) comprises: directing the vaporous second overhead stream (244b) to an evaporator (230), thereby condensing the second overhead stream (244b).

Clause 14: The method of any of clauses 1-19, wherein forming the condensed second overhead stream (270) comprises: directing the vaporous second overhead stream (244b) to a heat exchanger (810), thereby condensing the second overhead stream (244b).

Clause 15: The method of any of clauses 1-19, further comprising: directing (1650) at least a second portion of the condensed second overhead stream (270) to the separation system (135).

Clause 16: The method of any of clauses 1-19, wherein the first distillation column (202a) operates at a lower pressure than the second distillation column (202b).

Clause 17: The method of any of clauses 1-19, wherein the first distillation column (202a) operates at a higher pressure than the second distillation column (202b).

Clause 18: The method of any of clauses 1-19, wherein the rectification system (125) includes one of: a rectifier column (206) in direct fluid communication with the separation system (135) via a bottom stream (250) generated by the rectifier column (206); a rectifier/stripper column (310); and a rectifier column (206) in direct fluid communication with a side stripper (208) via the bottom stream (250).

Clause 19: The method of clauses 1-18, further comprising: recovering (1660) heat from a hot stream to heat a cold stream while generating the enriched solvent stream (268/440).

Clause 20: A method (1600), comprising: directing (1605) a first portion (242a) of a feed stream (240) comprising of an organic solvent, water, and solids to a first distillation column (202a) and a second portion (242b) of the feed stream (240) to a second distillation column (202b) operating at a different pressure than the first distillation column (202a), wherein the organic solvent is preferably an alcohol and more preferably ethanol; generating (1610a), in the first distillation column (202a), a vaporous first overhead stream (244a); generating (1610b), in the second distillation column (202b), a vaporous second overhead stream (244b); forming (1645) a condensed second overhead stream (270) from the vaporous second overhead stream (244b); directing (1625) the vaporous first overhead stream (244a) directly to a rectification system (125); generating (1630), via the rectification system (125), a third overhead stream (248); directing (1635) at least a portion the third overhead stream (248) to a separation system (135); and directing (1650) at least a portion of the condensed second overhead stream (270) to the separation system (135); and generating (1640), in the separation system (135), an enriched solvent stream (268/440).

Clause 21: The method of any of clauses 20-38, wherein the organic solvent is an alcohol, preferably ethanol.

Clause 22: The method of any of clauses 20-38, wherein the separation system (135) includes a membrane (212) and a vaporizer (510), wherein generating (1650) the enriched solvent stream (268/440) further comprises: contacting (1650a) the membrane (212) with a vapor stream (262) generated by the vaporizer (510), thereby generating a permeate stream (264); and directing (1650c) the permeate stream (264) to the one of the stripper column (210), the vaporizer (510), the rectification system (125), the first distillation column (202a), and the second distillation column (202b).

Clause 23: The method of any of clauses 20-38, wherein the separation system (135) includes a membrane (212) and one of a stripper column (210) or a vaporizer (510), wherein generating (1650) the enriched solvent stream (268/440) further comprises: contacting (1650a) the membrane (212) with a vapor stream (262) generated by the one of the stripper column (210) or the vaporizer (510), thereby generating a retentate stream (268); and directing (1650b) the retentate stream (268) to an evaporator (230) thereby forming a condensed retentate stream (270).

Clause 24: The method of any of clauses 20-38, wherein the separation system (135) includes a stripper column (210) and a membrane (212), wherein generating (1650) the enriched solvent stream (268/440) further comprises: contacting (1650a) the membrane (212) with a vapor stream (262) generated by the stripper column (210), thereby generating a permeate stream (264); and directing (1650c) the permeate stream (264) to the rectification system (125).

Clause 25: The method of any of clauses 20-38, wherein the separation system (135) includes a membrane (212) and one of a stripper column (210) and a vaporizer (510), wherein generating (1650) the enriched solvent stream (268/440) further comprises: contacting (1650a) the membrane (212) with a vapor stream (262/520) generated by the stripper column (210) or the vaporizer (510), thereby generating a retentate stream (268) and a permeate stream (264); directing (1650c) the permeate stream (264) to the second distillation column (202b); and directing (1650b) the retentate stream (268) to an evaporator (230).

Clause 26: The method of any of clauses 20-38, wherein the separation system (135) includes a membrane (212) and one of a stripper column (210) and a vaporizer (510), the method further comprising: contacting (1650a) the membrane (212) with a vapor stream (262/520) generated by the one of the stripper column (210) and the vaporizer (510), thereby generating a permeate stream (264); condensing the permeate stream (264) to form a condensed permeate stream (280); and directing (1650c) the condensed permeate stream (280) to at least one of the stripper column (210), the first distillation column (202a), the second distillation column (202b), and the rectification system (125).

Clause 27: The method of any of clauses 20-38, wherein the separation system (135) includes a molecular sieve unit (410) and one of a stripper column (210) or a vaporizer (510), wherein generating (1650) the enriched solvent stream (268/440) further comprises: contacting (1650a) the molecular sieve unit (410) with a vapor stream (262) generated by the one of the stripper column (210) or the vaporizer (510), thereby generating a regen stream (420); and directing (1650c) the permeate stream (264) to the one of the stripper column (210), the vaporizer (510), the rectification system (125), the first distillation column (202a), and the second distillation column (202b).

Clause 28: The method of any of clauses 20-38, wherein the separation system (135) includes a molecular sieve unit (410) and one of a stripper column (210) or a vaporizer (510), wherein generating (1650) the enriched solvent stream (268/440) further comprises: contacting (1650a) the molecular sieve unit (410) with a vapor stream (262) generated by the one of the stripper column (210) or the vaporizer (510), thereby generating a product stream (440); and directing (1650b) the product stream (440) to an evaporator (230) thereby forming a condensed product stream (440).

Clause 29: The method of any of clauses 20-38, wherein the separation system (135) includes a stripper column (210) and a molecular sieve unit (410), wherein generating (1650) the enriched solvent stream (268/440) further comprises: contacting (1650a) the molecular sieve unit (410) with a vapor stream (262) generated by the stripper column (210), thereby generating a regen stream (20); and directing (1650c) the regen stream (420) to the rectification system (125).

Clause 30: The method of any of clauses 20-38, wherein the separation system (135) includes a molecular sieve unit (410) and one of a stripper column (210) and a vaporizer (510), the method further comprising: contacting (1650a) the molecular sieve unit (410) with a vapor stream (262/520) generated by the one of the stripper column (210) and the vaporizer (510), thereby generating a regen stream (420); condensing the regen stream (420) to form a condensed regen stream (420); and directing (1650c) the condensed regen stream (420) to at least one of the stripper column (210) and the rectification system (125).

Clause 31: The method of any of clauses 20-38, wherein the separation system (135) includes: a membrane (212); a stripper column (210); a vaporizer (510); and a molecular sieve unit (410), the method further comprising: contacting (1650a) the molecular sieve unit (410) with a vapor stream (520) generated by the vaporizer (510), thereby generating a product stream (440); directing (1650b) the product stream (440) from the molecular sieve unit (410) to the stripper column (210) to generate a solvent-enriched overhead stream (262); contacting (1650a) the membrane (212) with the solvent-enriched overhead stream (262), thereby generating a retentate stream (268) having a higher concentration of the organic solvent than the solvent-enriched overhead stream (262).

Clause 32: The method of any of clauses 20-38, wherein forming the condensed second overhead stream (270) comprises: directing the vaporous second overhead stream (244b) to an evaporator (230), thereby condensing the second overhead stream (244b).

Clause 33: The method of any of clauses 20-38, wherein forming the condensed second overhead stream (270) comprises: directing the vaporous second overhead stream (244b) to a heat exchanger (810), thereby condensing the second overhead stream (244b).

Clause 34: The method of any of clauses 20-38, further comprising: directing (1650) at least a second portion of the condensed second overhead stream (270) to the separation system (135).

Clause 35: The method of any of clauses 20-38, wherein the first distillation column (202a) operates at a lower pressure than the second distillation column (202b).

Clause 36: The method of any of clauses 20-38, wherein the first distillation column (202a) operates at a higher pressure than the second distillation column (202b).

Clause 37: The method of any of clauses 20-38, wherein the rectification system (125) includes one of: a rectifier column (206) in direct fluid communication with the separation system (135) via a bottom stream (250) generated by the rectifier column (206); a rectifier/stripper column (310); and a rectifier column (206) in direct fluid communication with a side stripper (208) via the bottom stream (250).

Clause 38: The method of any of clauses 20-37, further comprising: directing (1650) at least a second portion of the condensed second overhead stream (270) to the rectification system (125).

Clause 39: A solvent production plant (100), comprising: a feed stripping section (110), including a first distillation column (202a) to generate a vaporous first overhead stream (244a) of an organic solvent, and a second distillation column (202b) to generate a vaporous second overhead stream (244b) of the organic solvent, wherein the first distillation column (202a) operates at a different pressure than the second distillation column (202b), and wherein the organic solvent is preferably an alcohol, and more preferably ethanol; a rectifying distillation section (120), including a rectification system (125) that directly receives the vaporous first overhead stream (244a) from the first distillation column (202a) to generate a third overhead stream (248); and a dehydration section (130), including a separation system (135) that receives at least a portion of the third overhead stream (248) to generate an enriched solvent stream (268/440), wherein the second distillation column (202b) is configured to direct the vaporous second overhead stream (244b) to at least one of the rectification system (125) and the separation system (135).

Clause 40: The solvent production plant any of clauses 39-59, wherein the rectification system (125) includes: a rectifier column (206) in direct fluid communication with the separation system (135) via a bottom stream (250) generated from the rectifier column (206).

Clause 41: The solvent production plant of any of claims 39-59, wherein the rectification system (125) includes: a rectifier/stripper column (310).

Clause 42: The solvent production plant of any of clauses 39-59, wherein the rectification system (125) includes: a rectifier column (206) in direct fluid communication with a side stripper (208) via a bottom stream (250) generated from the rectifier column (206), wherein the side stripper (208) directs a fourth overhead stream (252) back to the rectifier column (206).

Clause 43: The solvent production plant of any of clauses 39-58, wherein the separation system (135) includes: a stripper column (210) to generate a solvent-enriched overhead stream (262) from a solvent-water concentrated feed stream (286); and a membrane (212) to generate a permeate stream (264) and a retentate stream (268) from the solvent-enriched overhead stream (262).

Clause 44: The solvent production plant of any of clauses 39-59, wherein the separation system (135) includes: a stripper column (210) to generate a solvent-enriched overhead stream (262) from a solvent-water concentrated feed stream (286); and a molecular sieve unit (410) to generate a regen stream (420), a depressure stream (430), and a product stream (440) from the solvent-enriched overhead stream (262).

Clause 45: The solvent production plant of any of clauses 39-59, wherein the separation system (135) includes: a vaporizer (510) to generate a vaporized stream (520) from a first portion of a solvent-water concentrated feed stream (286); a molecular sieve unit (410) to generate a regen stream (420), a depressure stream (430), and a product stream (440) from the vaporized stream (520); a stripper column (210) to generate a solvent-enriched overhead stream (262) from the regen stream (420) and a second portion of the solvent-water concentrated feed stream (286); and a membrane (212) to generate a permeate stream (264) and a retentate stream (268) from the solvent-enriched overhead stream (262).

Clause 46: The solvent production plant of any of clauses 39-59, wherein the separation system (135) includes: a stripper column (210) to generate a solvent-enriched overhead stream (262) from a regen stream (420) and a second portion of a solvent-water concentrated feed stream (286); and a membrane (212) to generate the permeate stream (264) and a retentate stream (268) from the solvent-enriched overhead stream (262).

Clause 47: The solvent production plant of any of clauses 39-59, wherein the stripper column (210) further receives at least a portion of a redirected condensed second overhead stream (830) to generate the solvent-enriched overhead stream (262).

Clause 48: The solvent production plant of any of clauses 39-59, wherein the separation system (135) includes: a vaporizer (510) to generate a vaporized stream (520) from a solvent-water concentrated feed stream (286); and a membrane (212) to generate a permeate stream (264) and a retentate stream (268) from the vaporized stream (520).

Clause 49: The solvent production plant of any of clauses 39-59, wherein the separation system (135) includes: a vaporizer (510) to generate a vaporized stream (520) from a solvent-water concentrated feed stream (286); and a molecular sieve unit (410) to generate a regen stream (420), a depressure stream (430), and a product stream (440) from the vaporized stream (520).

Clause 50: The solvent production plant of any of clauses 39-59, wherein at a first stream of a first temperature is routed to exchange heat with a second stream of a second temperature, lower than the first temperature.

Clause 51: The solvent production plant of any of clauses 39-59, wherein at least a portion of a permeate stream (264) generated by the dehydration section (130) is routed as a vaporous input to the first distillation column (202a).

Clause 52: The solvent production plant of any of clauses 39-59, wherein at least a portion of a permeate stream (264) generated by the dehydration section (130) is routed as a vaporous input to the second distillation column (202b).

Clause 53: The solvent production plant of any of clauses 39-59, wherein at least a portion of a permeate stream (264) generated by the dehydration section (130) is routed as a vaporous input to the rectification system (125).

Clause 54: The solvent production plant of any of clauses 39-59, wherein at least a portion of a permeate stream (264) generated by the dehydration section (130) is routed as a condensed input to a side stripper (208) included in the rectification system (125).

Clause 55: The solvent production plant of any of clauses 39-59, wherein at least a portion of a permeate stream (264) generated by the dehydration section (130) is routed as a condensed input to a stripper column (210) included in the separation system (135).

Clause 56: The solvent production plant of any of clauses 39-59, wherein at least a portion of a permeate stream (264) generated by the dehydration section (130) is routed as a condensed input to the first distillation column (202a).

Clause 57: The solvent production plant of any of clauses 39-59, wherein at least a portion of a permeate stream (264) generated by the dehydration section (130) is routed as a condensed input to the second distillation column (202b).

Clause 58: The solvent production plant of any of clauses 39-58, wherein enriched solvent stream (268/440) is an alcohol, and more preferably 200-proof ethanol.

Clause 59: The solvent production plant of any of clauses 39-57, wherein the separation system (135) further receives at least a portion of the condensed second overhead stream (270) and a liquid permeate stream (280) recycled from the separation system (135) to generate the enriched solvent stream (268/440).

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Groupings of alternative elements or aspects of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects those of ordinary skill in the art to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific aspects disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Aspects of the invention so claimed are inherently or expressly described and enabled herein.

Further, it is believed that one skilled in the art can use the preceding description to use the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

1. A method, comprising:
directing a first portion of a feed stream comprising of an organic solvent, water, and solids to a first distillation column and a second portion of the feed stream to a second distillation column operating at a different pressure than the first distillation column, wherein the organic solvent is preferably an alcohol and more preferably ethanol;
generating, in the first distillation column, a vaporous first overhead stream;
directing the vaporous first overhead stream directly to a rectification system;
generating, in the second distillation column, a vaporous second overhead stream;
forming a condensed second overhead stream from the vaporous second overhead stream;
directing, at least a portion of the condensed second overhead stream to the rectification system;
generating, via the rectification system, a third overhead stream;
directing at least a portion of the third overhead stream to a separation system; and
generating, in the separation system, an enriched solvent stream.

2. The method of claim 1, wherein the separation system includes a membrane and a vaporizer, wherein generating the enriched solvent stream further comprises:
contacting the membrane with a vapor stream generated by the vaporizer, thereby generating a permeate stream; and
directing the permeate stream to the one of a stripper column, the vaporizer, the rectification system, the first distillation column, and the second distillation column.

3. The method of claim 1, wherein the separation system includes a membrane and one of a stripper column or a vaporizer, wherein generating the enriched solvent stream further comprises:
contacting the membrane with a vapor stream generated by the one of the stripper column or the vaporizer, thereby generating a retentate stream; and
directing the retentate stream to an evaporator thereby forming a condensed retentate stream.

4. The method of claim 1, wherein the separation system includes a stripper column and a membrane, wherein generating the enriched solvent stream further comprises:
contacting the membrane with a vapor stream generated by the stripper column, thereby generating a permeate stream; and
directing the permeate stream to the rectification system.

5. The method of claim 1, wherein the separation system includes a membrane and one of a stripper column and a vaporizer, wherein generating the enriched solvent stream further comprises:
contacting the membrane with a vapor stream generated by the stripper column or the vaporizer, thereby generating a retentate stream and a permeate stream;
directing the permeate stream to the second distillation column; and
directing the retentate stream to an evaporator.

6. The method of claim 1, wherein the separation system includes a membrane and one of a stripper column and a vaporizer, the method further comprising:
contacting the membrane with a vapor stream generated by the one of the stripper column and the vaporizer, thereby generating a permeate stream;
condensing the permeate stream to form a condensed permeate stream; and
directing the condensed permeate stream to at least one of the stripper column, the first distillation column, the second distillation column, and the rectification system.

7. The method of claim 1, wherein the separation system includes a molecular sieve unit and one of a stripper column or a vaporizer, wherein generating the enriched solvent stream further comprises:
contacting the molecular sieve unit with a vapor stream generated by the one of the stripper column or the vaporizer, thereby generating a regen stream; and
directing the regen stream to the one of the stripper column, the vaporizer, the rectification system, the first distillation column, and the second distillation column.

8. The method of claim 1, wherein the separation system includes a molecular sieve unit and one of a stripper column or a vaporizer, wherein generating the enriched solvent stream further comprises:
contacting the molecular sieve unit with a vapor stream generated by the one of the stripper column or the vaporizer, thereby generating a product stream; and
directing the product stream to an evaporator thereby forming a condensed product stream.

9. The method of claim 1, wherein the separation system includes a stripper column and a molecular sieve unit, wherein generating the enriched solvent stream further comprises:
contacting the molecular sieve unit with a vapor stream generated by the stripper column, thereby generating a regen stream; and
directing the regen stream to the rectification system.

10. The method of claim 1, wherein the separation system includes a molecular sieve unit and one of a stripper column and a vaporizer, the method further comprising:
contacting the molecular sieve unit with a vapor stream generated by the one of the stripper column and the vaporizer, thereby generating a regen stream;
condensing the regen stream to form a condensed regen stream; and
directing the condensed regen stream to at least one of the stripper column and the rectification system.

11. The method of claim 1, wherein the separation system includes:
a membrane;
a stripper column;
a vaporizer; and
a molecular sieve unit, the method further comprising:
contacting the molecular sieve unit with a vapor stream generated by the vaporizer, thereby generating a regen stream;
directing the regen stream from the molecular sieve unit to the stripper column to generate a solvent-enriched overhead stream; and
contacting the membrane with the solvent-enriched overhead stream, thereby generating a retentate stream having a higher concentration of the organic solvent than the solvent-enriched overhead stream.

12. The method of claim 1, wherein forming the condensed second overhead stream comprises:
directing the vaporous second overhead stream to an evaporator, thereby condensing the vaporous second overhead stream.

13. The method of claim 1, wherein forming the condensed second overhead stream comprises:
directing the vaporous second overhead stream to a heat exchanger, thereby condensing the second overhead stream.

14. The method of claim 1, further comprising:
directing at least a second portion of the condensed second overhead stream to the separation system.

15. The method of claim 1, wherein the first distillation column operates at a lower pressure than the second distillation column.

16. The method of claim 1, wherein the first distillation column operates at a higher pressure than the second distillation column.

17. The method of claim 1, wherein the rectification system includes one of:
a rectifier column in direct fluid communication with the separation system via a bottom stream generated by the rectifier column;
a rectifier/stripper column; and
a rectifier column in direct fluid communication with a side stripper via the bottom stream.

18. The method of claim 1, further comprising:
recovering heat from a hot stream to heat a cold stream while generating the enriched solvent stream.

19. A method, comprising:
directing a first portion of a feed stream comprising of an organic solvent, water, and solids to a first distillation column and a second portion of the feed stream to a second distillation column operating at a different pressure than the first distillation column, wherein the organic solvent is preferably an alcohol and more preferably ethanol;
generating, in the first distillation column, a vaporous first overhead stream;
generating, in the second distillation column, a vaporous second overhead stream;
forming a condensed second overhead stream from the vaporous second overhead stream;
directing the vaporous first overhead stream directly to a rectification system;
generating, via the rectification system, a third overhead stream;
directing at least a portion the third overhead stream to a separation system; and
directing at least a portion of the condensed second overhead stream to the separation system; and
generating, in the separation system, an enriched solvent stream.

20. The method of claim 19, further comprising:
directing at least a second portion of the condensed second overhead stream to the rectification system.

21-40. (canceled)
